(19) United States
(12) Patent Application Publication
(10) Pub. No.: US 2021/0353749 A1
(43) Pub. Date: Nov. 18, 2021

(54) GUANABENZ AS AN ADJUVANT FOR IMMUNOTHERAPY

(71) Applicant: UNIVERSITÉ CATHOLIQUE DE LOUVAIN, Louvain-la-Neuve (BE)

(72) Inventors: Benoît VAN DEN EYNDE, Rixensart (BE); Jingjing ZHU, Zaventem (BE)

(73) Assignee: UNIVERSITÉ CATHOLIQUE DE LOUVAIN, Louvain-la-Neuve (BE)

(21) Appl. No.: 17/286,913

(22) PCT Filed: Oct. 23, 2019
(86) PCT No.: PCT/EP2019/078867

§ 371 Date: Apr. 20, 2021

(30) Foreign Application Priority Data
Oct. 23, 2018 (EP) 18202122.0

(51) Int. Cl. A61K 39/39; A61K 31/155; A61K 39/00; A61P 35/00

(52) U.S. Cl. CPC A61K 39/39; A61P 35/00; A61K 39/0011; A61K 31/155

(57) ABSTRACT

Guanabenz for use with an immunotherapy in the treatment of a cancer or of an infectious disease. In particular, guanabenz is used as an adjuvant for an immunotherapy, such as a cancer immunotherapy or a vaccination. More specifically, guanabenz for use with an adoptive cell therapy, with a therapeutic vaccine, with a checkpoint inhibitor therapy or with a T-cell agonist therapy in the treatment of a cancer. Also, guanabenz for use with a vaccination in the prophylactic and/or therapeutic treatment of an infectious disease.

### FIELD OF INVENTION

The present invention relates to the field of immunotherapy, in particular to the field of cancer immunotherapy. More specifically, the present invention relates to the use of guanabenz as an adjuvant for an immunotherapy.

### BACKGROUND OF INVENTION

Immunotherapy can be broadly defined as a therapy aiming at inducing and/or enhancing an immune response towards a specific target, for example towards infectious agents such as viruses, bacteria, fungi or protozoan parasites, or towards cancer cells. In order to improve their therapeutic effects, immunotherapies are often administered in combination with an adjuvant. Adjuvant compounds thus seek to potentiate or modulate an immune response towards a specific target, in particular by enhancing, accelerating and/or prolonging said immune response.

In recent years, immunotherapy has proven to be one of the most promising developments in cancer treatment. Cancer immunotherapy manipulates a subject immune system with the aim of enhancing the immune response of the subject towards cancer cells, and thus of inducing the specific destruction of the cancer cells.

Currently, immunotherapy in cancer treatment can take many different forms, and includes for example the adoptive transfer of cells, notably of cytotoxic cells, the administration of checkpoint inhibitors, the administration of T-cell agonists, the administration of monoclonal antibodies or the administration of cytokines (Ribas & Wolchok, 2018, Science 359, 1350-1355; Galluzzi et al., 2014, Oncotarget 5, 12472-12508; Sharma & Allison, 2015, Science 348, 56-61). Immunotherapy in cancer treatment also includes therapeutic vaccines and the use of BCG (Bacillus Calmette-Guérin), the latter being used in the treatment of bladder cancer (Ribas & Wolchok, 2018, Science 359, 1350-1355; Garg et al., 2017, Trends Immunol 38, 577-593; Durgeau et al., 2018, Front Immunol 9, 14).

One of the central premises underlying cancer immunotherapy is the presence of antigens which are selectively or abundantly expressed or mutated in cancer cells, thus enabling the specific recognition and subsequent destruction of the cancer cells (Wirth & Kuhnel, 2017, Front Immunol 8, 1848; Hugo et al., 2016, Cell 165, 35-44, Coulie et al., 2014, Nature Reviews Cancer 14, 135-146). Another of the central premises underlying cancer immunotherapy is the presence of immune cells in the tumors, in particular of lymphocytes (Tumeh et al., 2014, Nature 515, 568-571). Such lymphocytes, commonly referred to as tumor infiltrating lymphocytes (TILs), notably comprise effector TILs which can target and kill the tumor cells through the recognition of the above-mentioned tumor-specific antigens (Durgeau et al., 2018, Front Immunol 9, 14; Tumeh et al., 2014, Nature 515, 568-571).

Yet, depending on the type of cancer and on the individual response, tumors are infiltrated to a varying degree with immune cells, and in particular with lymphocytes. Tumors with a high presence of lymphocytes are commonly referred to as “hot tumors”, while tumors with a low presence of lymphocytes are commonly referred to as “cold tumors” (Sharma & Allison, 2015, Science 348, 56-61).

It is known that increased effector T cell infiltration into tumors, and thus increased T cell response against the tumor cells, is correlated with increased survival for many different types of cancer. Thus, a number of cancer immunotherapies aim at increasing the infiltration and/or the activation of effector T cells within tumors.

One such immunotherapy consists in the transfer, i.e., infusion, of tumor targeting immune cells, such as tumor infiltrating T cells, to a subject. Such a transfer, referred to as an adoptive cell transfer, was first described in 1988 (Rosenberg et al., 1988, N Engl J Med 319, 1676-1680). Another such immunotherapy consists in the administration of a checkpoint inhibitor. Checkpoint inhibitors block interactions between inhibitory receptors expressed on T cells and their ligands. Checkpoint inhibitors are administered to prevent the inhibition of T cells by factors expressed by tumor cells and thus to enhance the T cell response against said tumor cells (Marin-Acevedo et al., 2018, J Hematol Oncol 11, 39).

However, the overall efficacy of immunotherapy remains limited in the majority of patients (Jenkins et al., 2018, Br J Cancer 118, 9-16; Ladanyi. 2015, Pigment Cell Melanoma Res 28, 490-500). One critical issue is the number of tumor-specific T cells present in the tumor and the exhaustion of said tumor infiltrating T cells, said exhaustion being characterized by a poor effector function, a sustained expression of inhibitory receptors and/or a transcriptional state distinct from that of functional effector or memory T cells (Jochems & Schlom, 2011, Exp Biol Med (Maywood) 236, 567-579).

Thus, there is a need for more effective immunotherapies, in particular more effective cancer immunotherapies. In particular, there is still a need for adjuvants to be administered with an immunotherapy, in particular with a cancer immunotherapy, said adjuvants potentiating the immunotherapy, notably by improving the cellular immune response against cancer cells, for example through an increase of T cells infiltration in the tumors, an increase of survival of the cancer-specific T cells and/or an increase of effector function of the cancer-specific T cells.

Guanabenz is a small molecule notably known as an alpha-2 adrenergic receptor agonist. Guanabenz (Wytensin®) was thus prescribed as an antihypertensive agent for oral administration. While seeking compounds able to potentiate the immune response towards cancer cells, the Applicant surprisingly showed that guanabenz is able to stimulate an immune response, notably a cellular immune response such as a T cell immune response. For example, the Applicant surprisingly found that guanabenz significantly increases the efficacy of a cancer immunotherapy by stimulating the functional activity of anti-tumor T cells and their ability to kill cancer cells in vivo. The Applicant also showed that guanabenz enhances the effects of a vaccination. Indeed, the Applicant surprisingly found that the administration of guanabenz with an antigen vaccine significantly enhances the specific cellular immune response induced by a reexposure to the antigen.

The present invention thus relates to guanabenz for use as an adjuvant for an immunotherapy. In particular, the present invention relates to guanabenz for use with an immunotherapy in the treatment of a cancer or an infectious disease. As illustrated hereinafter, guanabenz acts as an adjuvant for an immunotherapy, in particular for a cancer immunotherapy. Notably, the present invention relates to guanabenz for use with an adoptive cell therapy, with a CAR immune cell therapy, with a checkpoint inhibitor therapy, with a T-cell agonist therapy, with a therapeutic vaccination, with an antibody therapy (for example monoclonal antibodies and/or bispecific antibodies), with an oncolytic virus therapy, or with a cytokine therapy in the treatment of a cancer. The present invention also relates to guanabenz for use with a vaccination in the prophylactic and/or therapeutic treatment of an infectious disease.

### SUMMARY

The present invention relates to guanabenz for use with an immunotherapy in the treatment of a cancer or of an infectious disease in a subject in need thereof. In one embodiment, guanabenz is used as an adjuvant for the immunotherapy. In one embodiment, guanabenz is used as a conditioning regimen for the immunotherapy. In one embodiment, guanabenz is used as a conditioning regimen for the immunotherapy, a conditioning regimen being a therapy for preparing the subject for the immunotherapy.

In one embodiment, guanabenz is used with an immunotherapy in the treatment of a cancer selected from the group comprising or consisting acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma. In one embodiment, guanabenz is used with an immunotherapy in the treatment of a cancer selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma.

In one embodiment, guanabenz is used with an immunotherapy in the treatment of an infectious disease caused by a virus, a bacterium, a fungus or a protozoan parasite.

In one embodiment, guanabenz is to be administered prior to and/or concomitantly with the immunotherapy.

In one embodiment, guanabenz is to be administered at a dose ranging from about 0.01 mg per kilo body weight (mg/kg) to about 15 mg/kg.

According to one embodiment, the immunotherapy comprises an adoptive transfer of immune cells. In one embodiment, said immune cells are T cells or natural killer (NK) cells. In one embodiment, said immune cells are CAR T cells or CAR NK cells. In one embodiment, said immune cells are autologous immune cells. In one embodiment, said immune cells are CD8⁺ T cells.

According to one embodiment, the immunotherapy comprises a checkpoint inhibitor. In one embodiment, said checkpoint inhibitor is selected from the group comprising or consisting of inhibitors of PD-1 such as pembrolizumab, nivolumab, cemiplimab, tislelizumab, spartalizumab, ABBV-181 and JNJ-63723283, inhibitors of PD-L1 such as avelumab, atezolizumab and durvalumab, inhibitors of CTLA-4 such as ipilimumab and tremelimumab, and any mixtures thereof.

According to one embodiment, the immunotherapy comprises a vaccination.

### Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Adjuvant” in the present invention refers to a compound or a combination of compounds that potentiates an immunotherapy. In one embodiment, the adjuvant is used with an immunotherapy in the treatment of cancer and thus potentiates the immune response towards cancer cells. For example, an adjuvant may increase the number of lymphocytes, in particular tumor-infiltrated lymphocytes; increase the activation of lymphocytes, in particular tumor-infiltrated lymphocytes; increase the fitness of lymphocytes, in particular tumor-infiltrated lymphocytes; and/or increase the survival of lymphocytes, in particular tumor-infiltrated lymphocytes. In one embodiment, the adjuvant is used with an immunotherapy in the treatment of an infectious disease and thus potentiates the immune response towards an infectious agent. For example, an adjuvant may increase the number of lymphocytes, in particular effector lymphocytes; increase the activation of lymphocytes, in particular effector lymphocytes; increase the fitness of lymphocytes, in particular effector lymphocytes; and/or increase the survival of lymphocytes, in particular effector lymphocytes.
“Allogeneic” or “allogenic” refers to any material obtained or derived from a different subject of the same species than the subject to whom/which the material is to be introduced. Two or more subjects are said to be allogeneic to one another when the genes at one or more loci are not identical. In some aspects, allogeneic material from subjects of the same species may be sufficiently unlike genetically to interact antigenically.
“Autologous” refers to any material obtained or derived from the same subject to whom/which it is later to be re-introduced.
“Cancer immunotherapy” refers to an immunotherapy used for the treatment of a cancer, said immunotherapy modulating the immune response of a subject with the aim of inducing and/or stimulating the immune response of the subject towards cancer cells. In one embodiment, the cancer immunotherapy comprises or consists of the adoptive transfer of immune cells, in particular of T cells (such as alpha beta (αβ) T cells or gamma delta T cells), NK cells or NK T cells. In one embodiment, the cancer immunotherapy comprises or consists of the administration of a checkpoint inhibitor. In one embodiment, the cancer immunotherapy comprises or consists of the administration of a checkpoint agonist. In one embodiment, the cancer immunotherapy comprises or consists of the administration of an antibody. In one embodiment, the cancer immunotherapy comprises or consists of the administration of a therapeutic anti-cancer vaccine.
“Conditioning regimen” refers to a compound or therapy administered to prepare a subject for a subsequent therapy used in the treatment of a disease such as a cancer. For example, a conditioning regimen may be used before the adoptive transfer of immune cells.
“First-line therapy” also known as “primary therapy” or “induction therapy” refers to the first therapy administered for the treatment of a disease, for example a cancer. A first-line therapy may be completed or substituted with another therapy.
“Immunotherapy” refers to a therapy aiming at inducing and/or enhancing an immune response towards a specific target, for example towards infectious agents such as viruses, bacteria, fungi or protozoan parasites, or towards cancer cells. As used herein, examples of immunotherapies include, without being limited to, vaccination, such as preventive and therapeutic vaccination; adoptive transfer of immune cells, in particular of T cells (such as alpha beta (αβ) T cells or gamma delta T cells) or NK cells; checkpoint inhibitors; checkpoint agonists; antibodies.
“Infectious disease” refers to a disease caused by an infectious agent such as a virus, a bacterium, a fungus (for example a yeast), an alga, or a protozoan parasite (for example an amoeba).
“Pharmaceutically acceptable excipient” or “Pharmaceutically acceptable carrier” refers to an excipient or carrier commonly known and used in the field, including notably any and all solvents, dispersion media, coatings, antibacterial and antifungal agents, isotonic and absorption delaying agents. A pharmaceutically acceptable excipient or carrier thus refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (Food and Drug Administration) or EMA (European Medicines Agency).
“Pharmaceutically acceptable salt” refers to salts of a free acid or a free base which are not biologically undesirable and are generally prepared by reacting the free base with a suitable organic or inorganic acid or by reacting the free acid with a suitable organic or inorganic base. Suitable acid addition salts are formed from acids that form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen, phosphate/dihydrogen, phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemi-salts of acids and bases may also be formed, e.g. hemi-sulphate and hemi-calcium salts.
“Subject” refers to a mammal, preferably a human. In one embodiment, the subject is diagnosed with a cancer or with an infectious disease. In one embodiment, the subject is a patient, preferably a human patient, who/which is awaiting the receipt of, or is receiving, medical care or was/is/will be the subject of a medical procedure or is monitored for the development or progression of a disease, such as a cancer or an infectious disease. In one embodiment, the subject is a human patient who is treated and/or monitored for the development or progression of a cancer or an infectious disease. In one embodiment, the subject is a male. In another embodiment, the subject is a female. In one embodiment, the subject is an adult. In another embodiment, the subject is a child. In one embodiment, the subject is resistant to an immunotherapy. In one embodiment, the subject is resistant to a cancer immunotherapy.
“T cell immune response” refers to a T cell mediated immune response. In one embodiment, “T cell immune response” as used herein refers to an effector T cell mediated response, preferably a cytotoxic T cell mediated response. As used herein, “T cell immune response” includes immune responses mediated by alpha beta (αβ) T cells and immune responses mediated by gamma delta (γδ) T cells.
“Therapeutically effective amount” or “therapeutically effective dose” refers to the amount or dose of guanabenz that is aimed at, without causing significant negative or adverse side effects to the subject, (1) delaying or preventing the onset of a pathologic condition or disorder, in particular of a cancer or of an infectious disease in the subject; (2) reducing the severity or incidence of a pathologic condition or disorder, in particular of a cancer or of an infectious disease; (3) slowing down or stopping the progression, aggravation, or deterioration of one or more symptoms of a pathologic condition or disorder, in particular of a cancer or of an infectious disease affecting the subject; (4) bringing about ameliorations of the symptoms of a pathologic condition or disorder, in particular of a cancer or of an infectious disease affecting the subject; or (5) curing a pathologic condition or disorder affecting the subject, in particular a cancer or an infectious disease affecting the subject. A therapeutically effective amount may be administered prior to the onset of a pathologic condition or disorder, in particular of a cancer or an infectious disease, for a prophylactic or preventive action. Alternatively, or additionally, a therapeutically effective amount may be administered after initiation of a pathologic condition or disorder, in particular of a cancer or of an infectious disease, for a therapeutic action.
“Treating” or “treatment” refers to therapeutic treatment; to prophylactic or preventative measures; or to both, wherein the object is to prevent, slow down (lessen) or cure the targeted pathologic condition or disorder, e.g., a cancer or an infectious disease. In one embodiment of the present invention, “treating” or “treatment” refers to a therapeutic treatment. In another embodiment of the present invention, “treating” or “treatment” refers to a prophylactic or preventive treatment. In yet another embodiment of the present invention, “treating” or “treatment” refers to both a prophylactic (or preventive) treatment and a therapeutic treatment. Those in need of treatment include those already suffering from a pathologic condition or disorder, e.g. a cancer or an infectious disease, as well as those prone to develop a pathologic condition or disorder, e.g. a cancer or an infectious disease, or those in whom a pathologic condition or disorder, e.g. a cancer or an infectious disease, is to be prevented. In one embodiment, a subject suffering from a cancer or an infectious disease is successfully “treated” if, after receiving a therapeutically effective amount of guanabenz, in particular a therapeutically effective amount of guanabenz with an immunotherapy, the subject shows observable and/or measurable reduction in the number of cancer cells or in the number of infectious agents; reduction in the percent of total cells that are cancerous or in the percent of total cells that are infected; relief to some extent of one or more of the symptoms associated with the cancer or the infectious disease; reduced morbidity and mortality that is to say reduced risk of illness and/or death associated with the cancer or the infectious disease, and/or improvement in quality of life issues. The above parameters for assessing successful treatment and improvement in the disease are readily measurable by routine procedures familiar to a physician.
“Tumor infiltrating lymphocytes” or “TILs” refers to the T cells that are present in a tumor, either before an immunotherapy or after an immunotherapy, such as for example after an adoptive cell transfer or a therapeutic vaccination. As used herein, T cells encompass alpha beta (αβ) T cells and gamma delta (γδ) T cells. As used herein, T cells encompass CD4⁺ T cells and CD8⁺ T cells. As used herein, T cells also encompass T regulatory (Treg) cells, such as CD4⁺ Treg cells or CD8⁺ Treg cells, and T effector cells, such as CD4+ effector T cells and CD8+ effector T cells. In particular, CD8⁺ effector T cells include cytotoxic CD8⁺ T cells. In one embodiment, effector tumor infiltrating lymphocytes, or effector TILs, are the CD4⁺ or CD8⁺ effector T cells present in a tumor, either before an immunotherapy or after an immunotherapy, such as for example adoptive cell transfer or therapeutic vaccination. In one embodiment, regulatory tumor infiltrating lymphocytes, or regulatory TILs, are the CD4⁺ or CD8⁺ Treg cells present in a tumor, either before an immunotherapy or after an immunotherapy, such as for example an adoptive cell transfer or a therapeutic vaccination.
“Tumor-specific antigen” or “tumor-associated antigen” refers to an antigen specifically and/or abundantly expressed by cancer cells or tumor cells. T cells expressing T cell receptors recognizing and binding said antigens may be referred to as T cells recognizing a tumor-specific or tumor-associated antigen, T cells specific for a tumor-specific or tumor-associated antigen, T cells specific of a tumor-specific or tumor-associated antigen, or T cells directed to a tumor-specific or tumor-associated antigen.
“Vaccination” refers to the use of a preparation comprising a substance or a group of substances (i.e., a vaccine) meant to induce and/or enhance in a subject a targeted immune response towards an infectious agent (such as viruses, bacteria, fungi or protozoan parasites) or towards cancer cells. Prophylactic vaccination is used to prevent a subject from ever having a particular disease or to only have a mild case of the disease. For example, prophylactic vaccines may comprise the infectious agent responsible for an infectious disease (killed, inactivated, or live but weakened), or component(s) thereof (such as molecule(s) present at the surface of the infectious agent or toxin(s) secreted by the infectious agent) either isolated from the infectious agent or genetically engineered. Therapeutic vaccination is intended to treat a particular disease in a subject, for example cancers or infectious diseases such as herpes or hepatitis B. For example, therapeutic anti-cancer vaccines may comprise a tumor-associated antigen or tumor-associated antigens, aiming at inducing and/or enhancing a cell-mediated immune response, in particular a T cell immune response, directed towards the cancer cells expressing said tumor-associated antigen(s).

### DETAILED DESCRIPTION

The present invention relates to guanabenz for use in the treatment of diseases or conditions in which a modulation of the immune response is required.

In one embodiment, the present invention relates to guanabenz for use in the treatment of immune disorders in a subject in need thereof. In one embodiment, the present invention relates to guanabenz for use in the treatment of immune disorders in a subject in need thereof, said guanabenz being used as an immunomodulatory agent.

As used herein, “immune disorders” refers to diseases or conditions resulting from a malfunction of the immune system. Examples of immune disorders include, without being limited to, immunodeficiencies, autoimmune diseases, allergies, inflammatory diseases, asthma, and graft-versus-host disease (GVHD).

In particular, the present invention relates to guanabenz for use in the treatment of diseases or conditions in which an enhanced immune response is required.

In one embodiment, the present invention relates to guanabenz for use in the treatment of immunodeficiencies in a subject in need thereof. In one embodiment, the present invention relates to guanabenz for use in the treatment of immunodeficiencies in a subject in need thereof, said guanabenz being used for enhancing the immune response.

Examples of immunodeficiencies include, without being limited to, acquired immune deficiency syndrome (AIDS) and primary immunodeficiency diseases (PIs or PIDDs) also referred to as primary immunodeficiency disorders (PIDs) including X-linked agammaglobulinemia (XLA) and autosomal recessive agammaglobulinemia (ARA), ataxia telangiectasia, chronic granulomatous disease and other phagocytic cell disorders, common variable immune deficiency, complement deficiencies, DiGeorge syndrome, hemophagocytic lymphohistiocytosis (HLH), hyper IgE syndrome, hyper IgM syndromes, IgG subclass deficiency, innate immune defects, nuclear factor-kappa B essential modulator (NEMO) deficiency syndrome, selective IgA deficiency, selective IgM deficiency, severe combined immune deficiency and combined immune deficiency, specific antibody deficiency, transient hypogammaglobulinemia of infancy, WHIM syndrome (warts, hypogammaglobulinemia, infections, and myelokathexis), Wiskott-Aldrich syndrome.

In one embodiment, the present invention relates to guanabenz for use in the treatment of a cancer or of an infectious disease in a subject in need thereof, said guanabenz being used for enhancing an immune response towards the cancer cells or towards the infectious agent, respectively. In one embodiment, the present invention relates to guanabenz for use in the treatment of a cancer, wherein guanabenz is to be administered as a second-line therapy following an immunotherapy administered as a first-line therapy. In one embodiment, the present invention relates to guanabenz for use in the treatment of a cancer, wherein guanabenz is to be administered as a subsequent therapy following a previously administered immunotherapy.

The present invention also relates to guanabenz for use with an immunotherapy in the treatment of a cancer or of an infectious disease in a subject in need thereof. In one embodiment, the present invention relates to guanabenz for use with an immunotherapy in the treatment of a cancer or of an infectious disease in a subject in need thereof, said guanabenz being used as an adjuvant for the immunotherapy. In one embodiment, the present invention relates to guanabenz for use with an immunotherapy in the treatment of a cancer or of an infectious disease in a subject in need thereof, said guanabenz being used as a conditioning regimen for the immunotherapy.

The present invention also relates to an adjuvant for an immunotherapy for the treatment of a cancer or of an infectious disease comprising or consisting of guanabenz. In one embodiment, the present invention relates to an adjuvant for a cancer immunotherapy comprising or consisting of guanabenz. In one embodiment, the present invention relates to an adjuvant for a vaccination comprising or consisting of guanabenz.

The present invention also relates to a conditioning regimen for an immunotherapy for the treatment of a cancer or of an infectious disease comprising or consisting of guanabenz. In one embodiment, the present invention relates to a conditioning regimen for a cancer immunotherapy comprising or consisting of guanabenz. In one embodiment, the present invention relates to a conditioning regimen for a vaccination comprising or consisting of guanabenz.

The Applicant surprisingly showed that guanabenz is able to stimulate an immune response, notably a cellular immune response. In particular, the Applicant surprisingly showed that guanabenz significantly potentiates an immune response towards cancer cells, either when used alone or when use in combination with a cancer immunotherapy. As illustrated in the Examples hereinafter, in vitro incubation of T cells with guanabenz led to an increased T cell function as observed through an increased T cell degranulation and interferon gamma (IFNγ) secretion upon antigen recognition. Moreover, in vivo administration of guanabenz to mice, in particular when combined with an adoptive transfer of T cells, increased the infiltration and persistence of T cells in tumors, and also the activity of the tumor infiltrated T cells. The in vivo administration of guanabenz, in particular when combined with an adoptive transfer of T cells, thus resulted in an inhibition of tumor growth and an increase of survival. The Applicant also surprisingly showed that guanabenz enhances the effects of vaccination using irradiated tumor cells or ovalbumin protein. Indeed, the Applicant showed that guanabenz significantly potentiates the specific immune response, notably the T cell immune response, induced by the immunization. As illustrated in the Examples hereinafter, when mice were immunized using irradiated L1210 HA tumor cells or recombinant ovalbumin, the combined administration of guanabenz led to an increased immunization response as observed through an increased number of active CD8⁺ T cells in the spleen and blood of the immunized mice.

Guanabenz (CAS number 5051-62-7) is also known as 2-[(E)-(2,6-dichlorophenyl)methylideneamino]guanidine. Other names used to refer to guanabenz include 2-[(2,6-dichlorophenyl)methylideneamino]guanidine; N-(2,6-dichlorobenzylidene)-N′-amidinohydrazine; 2-((2,6-dichlorophenyl)methylene)hydrazinecarboximidamide; hydrazinecarboximidamide, 2-((2,6-dichlorophenyl)methylene)-; and WY-8678. Trade names of guanabenz include, without being limited to, Wytensin®, Wytens®, Lisapres® and Rexitene®. Guanabenz is also sometimes referred to as GBZ.

Guanabenz has the following formula:

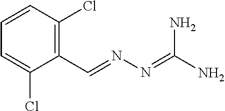 [SMILES: NC(N)=N/N=C/c1c(Cl)cccc1Cl]

As used herein, the term “guanabenz” encompasses any prodrugs, pharmaceutically acceptable salts, hydrates and solvates thereof. In particular, the term “guanabenz” encompasses the acetate and monoacetate salts thereof, e.g., guanabenz acetate and guanabenz monoacetate. The term “guanabenz” also encompasses the crystalline forms of said compounds.

Guanabenz was first described as an herbicidal compound in patent application GB1019120 published in 1966. Since, veterinary and medical uses of guanabenz have been studied, notably as a sedative or tranquilizer in animals and as an antihypertensive agent in humans. Guanabenz has thus been clinically used for a long time for the treatment of hypertension. Guanabenz is an agonist of the α2-adrenergic receptor and its antihypertensive effect is thought to be due to central alpha-adrenergic stimulation.

The present invention relates to guanabenz as described hereinabove for use with an immunotherapy in the treatment of a cancer or of an infectious disease in a subject in need thereof.

Another object of the present invention is a kit-of-parts comprising a first part comprising guanabenz and a second part comprising an immunotherapy for use in the treatment of a cancer or of an infectious disease in a subject in need thereof. In one embodiment, the kit-of-parts of the invention comprises a first part comprising guanabenz and a second part comprising an immunotherapy, such as, for example, a checkpoint inhibitor, for use in the treatment of a cancer in a subject in need thereof.

According to the present invention, an immunotherapy is defined as a therapy modulating the immune response of a subject with the aim of inducing and/or enhancing an immune response towards a specific target.

In one embodiment, the immunotherapy comprises or consists of an adoptive cell therapy, in particular an adoptive T cell therapy, an adoptive NK cell therapy and/or a CAR immune cell therapy, a checkpoint inhibitor therapy, a T-cell agonist therapy, a vaccination, such as a preventive vaccination or a therapeutic vaccination, an antibody therapy, a cytokine therapy or any mixes thereof.

In one embodiment, the immunotherapy comprises or consists of an adoptive cell therapy, in particular an adoptive T cell therapy, an adoptive NK cell therapy and/or a CAR immune cell therapy, a checkpoint inhibitor therapy, a vaccination, such as a preventive vaccination or a therapeutic vaccination, an antibody therapy, or any mixes thereof.

In one embodiment, the immunotherapy comprises or consists of an adoptive cell therapy, in particular an adoptive T cell therapy, an adoptive NK cell therapy and/or a CAR immune cell therapy, a checkpoint inhibitor therapy, a vaccination, such as a preventive vaccination or a therapeutic vaccination, or any mixes thereof.

In one embodiment, the immunotherapy comprises or consists of an adoptive cell therapy, in particular an adoptive T cell therapy or an adoptive NK cell therapy, a checkpoint inhibitor therapy, a vaccination, such as a preventive vaccination or a therapeutic vaccination, or any mixes thereof.

In one embodiment, the immunotherapy comprises or consists of an adoptive cell therapy, in particular an adoptive T cell therapy, an adoptive NK cell therapy and/or a CAR immune cell therapy, a vaccination, such as a preventive vaccination or a therapeutic vaccination, or any mixes thereof.

In one embodiment, the immunotherapy comprises or consists of an adoptive cell therapy, in particular an adoptive T cell therapy or an adoptive NK cell therapy, or a vaccination, such as a preventive vaccination or a therapeutic vaccination.

According to one embodiment, the present invention relates to guanabenz for use with an immunotherapy in the treatment of a cancer in a subject in need thereof. Thus, in one embodiment, the present invention relates to guanabenz for use with a cancer immunotherapy in the treatment of a cancer in a subject in need thereof.

According to the present invention, an immunotherapy as a cancer treatment, i.e., a cancer immunotherapy, is defined as a therapy modulating the immune response of a subject with the aim of inducing and/or enhancing the immune response of the subject towards cancer cells.

One of the central premises underlying cancer immunotherapy is the presence of antigens which are selectively or abundantly expressed or mutated in cancer cells, thus enabling the specific recognition and subsequent destruction of the cancer cells. Such antigens are commonly referred to as tumor-specific antigens. Another of the central premises underlying cancer immunotherapy is the presence of lymphocytes in the tumors, i.e., tumor infiltrating lymphocytes (TILs), and notably of effector TILs which can target and kill the tumor cells through the recognition of the above-mentioned tumor-specific antigens.

Examples of cancer immunotherapies include, without being limited to, adoptive transfer of immune cells; checkpoint inhibitors; T-cell agonists also referred to as checkpoint agonists; antibodies including monoclonal antibodies, antibody domains, antibody fragments, bispecific antibodies; cytokines; oncolytic viruses; preventive and therapeutic vaccines, BCG (Bacillus Calmette-Guérin); immunotherapies relying on ARN therapies, such as, for example immune cells modified ex vivo by RNA interference (also known as RNAi) or RNA-based vaccines.

In one embodiment, the immunotherapy used for the treatment of a cancer with guanabenz as described hereinabove comprises or consists of an adoptive cell therapy, in particular an adoptive T cell therapy or an adoptive NK cell therapy, a CAR immune cell therapy, a checkpoint inhibitor therapy, a T-cell agonist therapy, a therapeutic vaccination, an antibody therapy, an oncolytic virus therapy, a cytokine therapy or any mixes thereof.

According to one embodiment, the immunotherapy used for the treatment of a cancer with guanabenz as described hereinabove comprises or consists of an adoptive transfer of cells, also referred to as adoptive cell therapy (both also referred to as ACT), particularly an adoptive transfer of T cells or NK cells, also referred to as adoptive T cell therapy or adoptive NK cell therapy, respectively. Thus, in one embodiment, the immunotherapy used for the treatment of a cancer with guanabenz as described hereinabove is an adoptive cell therapy, in particular an adoptive T cell therapy or an adoptive NK cell therapy.

As used herein, an adoptive transfer of cells or adoptive cell therapy is defined as the transfer, for example as an infusion, of immune cells to a subject. As a cancer treatment, the adoptive transfer of immune cells to a subject aims at enhancing the subject immune response towards the cancer cells.

In one embodiment, the transferred immune cells are T cells or natural killer (NK) cells. In one embodiment, the transferred immune cells are T cells, in particular CD8+ T cells, and/or natural killer (NK) cells.

In one embodiment, the transferred immune cells are cytotoxic cells. Examples of cytotoxic cells include natural killer (NK) cells, CD8⁺ T cells, and natural killer (NK) T cells.

In one embodiment, the transferred immune cells are natural killer (NK) cells.

In one embodiment, the transferred immune cells are T cells, in particular effector T cells.

Examples of effector T cells include CD4⁺ T cells and CD8⁺ T cells.

In one embodiment, the transferred immune cells are alpha beta (αβ) T cells. In another embodiment, the transferred immune cells are gamma delta (γδ) T cells.

In one embodiment, the transferred immune cells are CD4⁺ T cells, CD8⁺ T cells, or natural killer (NK) T cells, preferably the transferred T cells are CD8⁺ T cells.

In one embodiment, the transferred immune cells as described hereinabove are antigen-specific immune cells. In one embodiment, the transferred immune cells as described hereinabove are antigen-specific immune cells, wherein said antigen is specifically and/or abundantly expressed by cancer cells. In one embodiment, the transferred immune cells as described hereinabove are tumor-specific immune cells, in other words the transferred immune cells as described hereinabove specifically recognize cancer cells or tumor cells through an antigen specifically and/or abundantly expressed by said cancer cells or tumor cells. In one embodiment, the transferred immune cells as described hereinabove are tumor-specific effector T cells. In one embodiment, the transferred immune cells as described hereinabove are tumor-specific CD8⁺ effector T cells, in particular tumor-specific cytotoxic CD8⁺ T cells. In one embodiment, the transferred immune cells as described hereinabove are tumor-specific cytotoxic cells. In one embodiment, the transferred immune cells as described hereinabove are tumor-specific NK cells.

Examples of tumor-specific antigens, i.e., antigens that are specifically and/or abundantly expressed by cancer cells include, without being limited to, neoantigens (also referred to as new antigens or mutated antigens), 9D7, ART4, β-catenin, BING-4, Bcr-abl, BRCA1/2, calcium-activated chloride channel 2, CDK4, CEA (carcinoembryonic antigen), CML66, Cyclin B1, CypB, EBV (Epstein-Barr virus) associated antigens (such as LMP-1, LMP-2, EBNA1 and BARF1), EGFRvIII, Ep-CAM, EphA3, fibronectin, Gp100/pmel17, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-2, p53, PRAME, prostate-specific antigen (PSA), PSMA (prostate-specific membrane antigen), Ras, SAP-1, SART-I, SART-2, SART-3, SSX-2, survivin, TAG-72, telomerase, TGF-βRII, TRP-1/-2, tyrosinase, WT1, antigens of the BAGE family, antigens of the CAGE family, antigens of the GAGE family, antigens of the MAGE family, antigens of the SAGE family, and antigens of the XAGE family.

As used herein, neoantigens (also referred to as new antigens or mutated antigens) correspond to antigens derived from proteins that are affected by somatic mutations or gene rearrangements acquired by the tumors. Neoantigens may be specific to each individual subject and thus provide targets for developing personalized immunotherapies. Examples of neoantigens include for example, without being limited to, the R24C mutant of CDK4, the R24L mutant of CDK4, KRAS mutated at codon 12, mutated p53, the V600E mutant of BRAF and the R132H mutant of IDH1.

In one embodiment, the transferred immune cells as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs corresponds to antigens encoded by genes that are expressed in tumor cells but not in normal tissues except in male germline cells. Examples of CTAs include, without being limited to, MAGE-AL MAGE-A3, MAGE-A4, MAGE-C2, NY-ESO-1, PRAME and SSX-2.

As used herein, the class of viral antigens corresponds to antigens derived from viral oncogenic proteins. Examples of viral antigens include, without being limited to, HPV (human papillomavirus) associated antigens such as E6 and E7, and EBV (Epstein-Barr virus) associated antigens such as LMP-1, LMP-2, EBNA1 and BARF1.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

For example, autologous T cells can be generated ex vivo either by expansion of antigen-specific T cells isolated from the subject or by redirection of T cells of the subject through genetic engineering.

In one embodiment, the immune cells to be infused are modified ex vivo, in particular with RNA interference (also known as RNAi), before being infused to the subject.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In one embodiment, the immunotherapy used for the treatment of a cancer with guanabenz as described hereinabove comprises or consists of a CAR immune cell therapy, in particular a CAR T cell therapy or a CAR NK cell therapy. Thus, in one embodiment, the immunotherapy used for the treatment of a cancer with guanabenz as described hereinabove is a CAR immune cell therapy, in particular a CAR T cell therapy or a CAR NK cell therapy.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

Examples of tumor-specific antigens are mentioned hereinabove.

In one embodiment, the transferred CAR T cells or CAR NK cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, EphA2, CD19, CD21, and CD20.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

According to one embodiment, the immunotherapy used for the treatment of a cancer with guanabenz as described hereinabove comprises or consists of at least one checkpoint inhibitor. Thus, in one embodiment, the immunotherapy used for the treatment of a cancer with guanabenz as described hereinabove is a checkpoint inhibitor therapy.

As used herein, a checkpoint inhibitor therapy is defined as the administration of at least one checkpoint inhibitor to the subject.

Checkpoint inhibitors (CPI, that may also be referred to as immune checkpoint inhibitors or ICI) block the interactions between inhibitory receptors expressed on T cells and their ligands. As a cancer treatment, checkpoint inhibitor therapy aims at preventing the activation of inhibitory receptors expressed on T cells by ligands expressed by the tumor cells. Checkpoint inhibitor therapy thus aims at preventing the inhibition of T cells present in the tumor, i.e., tumor infiltrating T cells, and thus at enhancing the subject immune response towards the tumor cells.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of IDO (indoleamine 2,3-dioxygenase) and inhibitors of TDO (tryptophan 2,3-dioxygenase); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

In one embodiment, the at least one checkpoint inhibitor is selected from the group comprising or consisting of inhibitors or PD-1, inhibitors of PD-L1, inhibitors of CTLA-4 and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, avelumab, atezolizumab, durvalumab, LY3300054, ipilimumab, tremelimumab, and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, avelumab, atezolizumab, durvalumab, ipilimumab, tremelimumab, and any mixtures thereof.

In one embodiment, the at least one checkpoint inhibitor is an inhibitor of PD-1, also referred to as an anti-PD-1.

Inhibitors of PD-1 may include antibodies targeting PD-1, in particular monoclonal antibodies, and non-antibody inhibitors such as small molecule inhibitors.

Examples of inhibitors of PD-1 include, without being limited to, pembrolizumab, nivolumab, cemiplimab, tislelizumab, spartalizumab, ABBV-181, JNJ-63723283, BI 754091, MAG012, TSR-042, and AGEN2034.

Pembrolizumab is also known as MK-3475, MK03475, lambrolizumab, or SCH-900475. The trade name of pembrolizumab is Keytruda®.

Nivolumab is also known as ONO-4538, BMS-936558, MDX1106, or GTPL7335. The trade name of nivolumab is Opdivo®.

Cemiplimab is also known as REGN2810 or REGN-2810.

Tislelizumab is also known as BGB-A317.

Spartalizumab is also known as PDR001 or PDR-001.

In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, and any mixtures thereof.

In one embodiment, the at least one checkpoint inhibitor is an inhibitor of PD-L1, also referred to as an anti-PD-L1.

Inhibitors of PD-L1 may include antibodies targeting PD-L1, in particular monoclonal antibodies, and non-antibody inhibitors such as small molecule inhibitors.

Examples of inhibitors of PD-L1 include, without being limited to, avelumab, atezolizumab, durvalumab and LY3300054.

Avelumab is also known as MSB0010718C, MSB-0010718C, MSB0010682, or MSB-0010682. The trade name of avelumab is Bavencio®.

Atezolizumab is also known as MPDL3280A (clone YW243.55.S70), MPDL-3280A, RG-7446 or RG7446. The trade name of atezolizumab is Tecentriq®.

Durvalumab is also known as MEDI4736 or MEDI-4736. The trade name of durvalumab is Imfinzi®.

In one embodiment, the at least one checkpoint inhibitor is selected from the group comprising or consisting of avelumab, atezolizumab, durvalumab, LY3300054, and any mixtures thereof.

In one embodiment, the at least one checkpoint inhibitor is selected from the group comprising or consisting of avelumab, atezolizumab, durvalumab, and any mixtures thereof.

In one embodiment, the at least one checkpoint inhibitor is an inhibitor of CTLA-4, also referred to as an anti-CTLA-4.

Inhibitors of CTLA-4 may include antibodies targeting CTLA-4, in particular monoclonal antibodies, and non-antibody inhibitors such as small molecule inhibitors.

Examples of inhibitors of CTLA-4 include, without being limited to, ipilimumab and tremelimumab.

Ipilimumab is also known as BMS-734016, MDX-010, or MDX-101. The trade name of ipilimumab is Yervoy®.

Tremelimumab is also known as ticilimumab, CP-675, or CP-675,206.

In one embodiment, the at least one checkpoint inhibitor is selected from the group comprising or consisting of ipilimumab, tremelimumab, and any mixtures thereof.

In one embodiment, the at least one checkpoint inhibitor is an inhibitor of IDO or an inhibitor of TDO, also referred to as an anti-IDO or anti-TDO, respectively.

Examples of inhibitors of IDO include, without being limited to, 1-methyl-D-tryptophan (also known as indoximod), epacadostat (also known as INCB24360), navoximod (also known as IDO-IN-7 or GDC-0919), linrodostat (also known as BMS-986205), PF-06840003 (also known as EOS200271), TPST-8844, and LY3381916.

According to one embodiment, the immunotherapy used for the treatment of a cancer with guanabenz as described hereinabove comprises or consists of at least one T-cell agonist (sometimes also referred to as checkpoint agonist). Thus, in one embodiment, the immunotherapy used for the treatment of a cancer with guanabenz as described hereinabove is a T-cell agonist therapy.

As used herein, a T-cell agonist therapy is defined as the administration of at least one T-cell agonist to the subject.

T-cell agonists act by activating stimulatory receptors expressed on immune cells, such as T cells. As used herein, the term “stimulatory receptors” refers to receptors that induce a stimulatory signal upon activation, and thus lead to an enhancement of the immune response. As a cancer treatment, T-cell agonist therapy aims at activating stimulatory receptors expressed on immune cells present in a tumor. In particular, T-cell agonist therapy aims at enhancing the activation of T cells present in a tumor, i.e., tumor infiltrating T cells, and thus at enhancing the subject immune response towards the tumor cells. Currently, a number of potential targets for T-cell agonist therapy have been identified.

Examples of T-cell agonists include, without being limited to, agonists of CD137 (cluster differentiation 137) also known as 4-1BB or TNFRS9 (tumor necrosis factor receptor superfamily, member 9); agonists of OX40 receptor also known as CD134 (cluster differentiation 134) or TNFRSF4 (tumor necrosis factor receptor superfamily, member 4); agonists of GITR (glucocorticoid-induced TNF receptor family-related protein); agonists of ICOS (inducible co-stimulator); agonists of CD27-CD70 (cluster differentiation 27-cluster differentiation 70); and agonists of CD40 (cluster differentiation 40).

In one embodiment, the at least one T-cell agonist is selected from the group comprising or consisting of agonists of CD137, agonists of OX40, agonists of GITR, agonists of ICOS, agonists of CD27-CD70, agonists of CD40 and any mixtures thereof.

Examples of agonists of CD137 include, without being limited, utomilumab and urelumab.

According to one embodiment, the immunotherapy used for the treatment of a cancer with guanabenz as described hereinabove comprises or consists of a vaccine. Thus, in one embodiment, the immunotherapy used for the treatment of a cancer with guanabenz as described hereinabove is a vaccination.

According to one embodiment, the immunotherapy used for the treatment of a cancer with guanabenz as described hereinabove comprises or consists of a therapeutic vaccine (sometimes also referred to as a treatment vaccine). Thus, in one embodiment, the immunotherapy used for the treatment of a cancer with guanabenz as described hereinabove is a therapeutic vaccination.

As used herein, a therapeutic vaccine is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; RNA-based vaccines, and dendritic cell vaccines.

According to one embodiment, the immunotherapy used for the treatment of a cancer with guanabenz as described hereinabove comprises or consists of an antibody therapy. Thus, in one embodiment, the immunotherapy used for the treatment of a cancer with guanabenz as described hereinabove is an antibody therapy.

As used herein, an antibody therapy is defined as the administration of at least one antibody to the subject.

As a cancer treatment, antibody therapy aims at enhancing the subject immune response towards the cancer cells, notably by targeting cancer cells for destruction, by stimulating the activation of T cells present in the tumor or by preventing the inhibition of T cells present in the tumor, or at inhibiting the growth or spreading of cancer cells.

As used herein, “antibody therapy” may include the administration of monoclonal antibodies, polyclonal antibodies, multiple-chain antibodies, single-chain antibodies, single-domain antibodies, antibody fragments, antibody domains, antibody mimetics or multi-specific antibodies such as bispecific antibodies.

In one embodiment, the antibody is for or aims at targeting cancer cells or tumor cells for destruction.

Examples of antibodies, in particular monoclonal antibodies, targeting cancer cells or tumor cells for destruction include tumor-specific antibodies, in particular tumor-specific monoclonal antibodies. Examples of tumor-specific antibodies, include, without being limited to, antibodies targeting cell surface markers of cancer cells or tumor cells, antibodies targeting proteins involved in the growth or spreading of cancer cells or tumor cells.

In one embodiment, the antibody is for or aims at stimulating the activation of T cells present in the tumor.

Examples of antibodies, in particular monoclonal antibodies, stimulating the activation of T cells present in the tumor include, without being limited to, anti-CD137 antibodies and anti-OX40 antibodies as described hereinabove.

In one embodiment, the antibody is for or aims at preventing the inhibition of T cells present in the tumor.

Examples of antibodies, in particular monoclonal antibodies, preventing the inhibition of T cells present in the tumor include, without being limited to, anti-PD-1 antibodies (such as pembrolizumab, nivolumab, cemiplimab, tislelizumab, and spartalizumab), anti-PD-L1 antibodies (such as avelumab, atezolizumab, and durvalumab) and anti-CTLA-4 antibodies (such as ipilimumab and tremelimumab) as described hereinabove.

In one embodiment, the antibody is for or aims at inhibiting the growth or spreading of cancer cells.

Examples of antibodies inhibiting the growth or spreading of cancer cells include, without being limited to, anti-HER2 antibodies (such as trastuzumab).

According to one embodiment, the immunotherapy used for the treatment of a cancer with guanabenz as described hereinabove comprises or consists of an oncolytic virus therapy. Thus, in one embodiment, the immunotherapy used for the treatment of a cancer with guanabenz as described hereinabove is an oncolytic virus therapy.

As used herein, an oncolytic virus therapy is defined as the administration of at least one oncolytic virus to the subject.

Oncolytic viruses are defined as viruses that preferentially infect and kill cancer cells over normal, non-cancer, cells. As a cancer treatment, oncolytic virus therapy aims at killing cancer cells and/or triggering or enhancing an immune response towards the cancer cells.

Examples of oncolytic viruses include, without being limited to, modified herpes simplex type-1 viruses such as talimogene laherparepvec (also known as T-VEC) or HSV-1716; modified adenoviruses such as Ad5-DNX-2401; modified measles viruses such as MV-NIS; modified vaccinia viruses (VV) such as vaccinia virus TG6002; and modified polioviruses such as PVS-RIPO.

According to one embodiment, the immunotherapy used for the treatment of a cancer with guanabenz as described hereinabove comprises or consists of a cytokine therapy. Thus, in one embodiment, the immunotherapy used for the treatment of a cancer with guanabenz as described hereinabove is a cytokine therapy.

As used herein, a cytokine therapy is defined as the administration of at least one cytokine, in particular a recombinant cytokine, to the subject.

As a cancer treatment, cytokine therapy aims at enhancing the subject immune response towards the cancer cells, notably by stimulating the activation of immune cells.

Examples of cytokines that may be administered as a cytokine therapy include, without being limited to, interleukin-2 (IL-2) and interferon-alpha (IFN-α).

According to one embodiment, the present invention relates to guanabenz for use with an immunotherapy in the treatment of an infectious disease in a subject in need thereof.

According to the present invention, an immunotherapy as a treatment for an infectious disease is defined as a therapy modulating the immune response of a subject with the aim of inducing and/or enhancing the immune response of the subject towards the infectious agent responsible for the infectious disease.

Examples of immunotherapies used in the treatment of an infectious disease include, without being limited to, preventive vaccines, therapeutic vaccines, monoclonal antibodies, cytokines, the adoptive transfer of T cells, granulocyte transfusions, and checkpoint inhibitors.

In one embodiment, the immunotherapy used for the treatment of an infectious disease with guanabenz as described hereinabove comprises or consists of a preventive vaccination, a therapeutic vaccination, an adoptive T cell therapy, an antibody therapy, or any mixes thereof.

According to one embodiment, the immunotherapy used for the treatment of an infectious disease with guanabenz as described hereinabove comprises or consists of a vaccine, including a preventive vaccine or a therapeutic vaccine. Thus, in one embodiment, the immunotherapy used for the treatment of an infectious disease with guanabenz as described hereinabove is a vaccination, in particular a preventive vaccination or a therapeutic vaccination.

The present invention relates to guanabenz for use as an adjuvant for an immunotherapy as described hereinabove, in particular for a cancer immunotherapy as described hereinabove or a vaccination as described hereinabove.

Thus, according to the present invention, guanabenz as described hereinabove is used as an adjuvant for an immunotherapy, in particular for a cancer immunotherapy or a vaccination. In other words, according to the present invention, guanabenz as described hereinabove potentiates an immunotherapy, in particular a cancer immunotherapy or a vaccination.

In one embodiment, potentiation of an immunotherapy in the presence of an adjuvant, in particular of a cancer immunotherapy or a vaccination, is defined by comparison with an immunotherapy, in particular a cancer immunotherapy or a vaccination, administered alone.

In one embodiment, said potentiation by an adjuvant, i.e., guanabenz, of a cancer immunotherapy, is defined as at least one of the following, observed in the subject recipient of said cancer immunotherapy:
increase of the number of lymphocytes (e.g., cytotoxic CD8⁺ T cells or NK cells), in particular of tumor-infiltrated effector lymphocytes;
increase in the activation of lymphocytes (e.g., cytotoxic CD8⁺ T cells or NK cells), in particular of tumor-infiltrated effector lymphocytes;
increase in the fitness of lymphocytes (e.g., cytotoxic CD8⁺ T cells or NK cells), in particular of tumor-infiltrated effector lymphocytes, wherein fitness is assessed as the TCR-triggered signaling, proliferation and/or cytokine production by said lymphocytes and/or as the survival of said lymphocytes;
increase in the survival or persistence of lymphocytes (e.g., cytotoxic CD8⁺ T cells or NK cells), in particular of tumor-infiltrated effector lymphocytes;
decrease of the number of suppressive immune cells, such as suppressive myeloid cells (for example MDSCs and/or tumor-associated macrophages) and/or suppressive lymphocytes (for example T regulatory cells), in particular of tumor-infiltrated suppressive immune cells;
decrease in the activation of suppressive immune cells, such as suppressive myeloid cells (for example MDSCs and/or tumor-associated macrophages) and/or suppressive lymphocytes (for example T regulatory cells), in particular of tumor-infiltrated suppressive immune cells;
decrease in the fitness of suppressive immune cells, such as suppressive myeloid cells (for example MDSCs and/or tumor-associated macrophages) and/or suppressive lymphocytes (for example T regulatory cells), in particular of tumor-infiltrated suppressive immune cells, wherein fitness is assessed as the activation, proliferation and/or cytokine production by said suppressive immune cells, and/or as the survival of said suppressive immune cells;
decrease in the survival of suppressive immune cells, such as suppressive myeloid cells (for example MDSCs and/or tumor-associated macrophages) and/or suppressive lymphocytes (for example T regulatory cells), in particular of tumor-infiltrated suppressive immune cells;
decrease in the tumor growth and/or in the tumor size; and/or increase in survival.

The above-listed parameters are well-known to the person skilled in the art. Moreover, methods to determine the number, activation, fitness and/or survival of lymphocytes, such as T cells or NK cells, are commonly used in the field. Such methods include, for example, FACS analysis conducted on a sample, in particular a tumor sample, obtained from a subject (see for example Zhu et al., 2017, Nat Commun 8, 1404).

In one embodiment, said potentiation by an adjuvant, i.e., guanabenz, of a vaccination, is defined as at least one of the following, observed in the subject recipient of said vaccination:
increase of the number of effector lymphocytes such as, for example, cytotoxic CD8⁺ T cells, in particular of specific effector lymphocytes;
increase in the activation of effector lymphocytes such as, for example, cytotoxic CD8+ T cells, in particular of specific effector lymphocytes;
increase in the amount of antigen-specific antibodies.

The above-listed parameters are well-known to the person skilled in the art. Moreover, methods to determine the number, activation, survival of lymphocytes are commonly used in the field. Such methods include, for example, FACS analysis conducted on a sample, for example a blood or a tumor sample, obtained from a subject.

According to the present invention, guanabenz is to be administered either simultaneously, separately or sequentially with respect to an immunotherapy, in particular a cancer immunotherapy or a vaccination, for which it is used as an adjuvant.

The present invention also relates to guanabenz for use as a conditioning regimen for a subsequent immunotherapy as described hereinabove, in particular a cancer immunotherapy as described hereinabove (in other words, guanabenz for use as a conditioning regimen is for preparing the subject for a subsequent immunotherapy, in particular a cancer immunotherapy).

In one embodiment, guanabenz is thus to be administered prior to an immunotherapy, in particular an adoptive cell therapy, a checkpoint inhibitor therapy or a vaccination. In one embodiment, guanabenz is to be administered prior to and concomitantly with an immunotherapy, in particular an adoptive cell therapy, a checkpoint inhibitor therapy or a vaccination. In one embodiment, guanabenz is to be administered prior to an immunotherapy, in particular an adoptive cell therapy, a checkpoint inhibitor therapy or a vaccination, and continuously thereafter.

Another object of the present invention is a method for modulating an immune response, in particular a cellular immune response, in a subject in need thereof, said method comprising administering to the subject guanabenz as described hereinabove.

According to one embodiment, said method for modulating an immune response, in particular a cellular immune response, comprises administering to the subject a therapeutically effective dose of guanabenz.

Another object of the present invention is a method for stimulating or enhancing an immune response, in particular a cellular immune response, in a subject in need thereof, said method comprising administering to the subject guanabenz as described hereinabove.

According to one embodiment, said method for stimulating or enhancing an immune response, in particular a cellular immune response, comprises administering to the subject a therapeutically effective dose of guanabenz.

In one embodiment, the immune response, in particular the cellular immune response, is a T cell response or a NK cell response. In one embodiment, the T cell response is an alpha beta (αβ) T cell response or a gamma delta (γδ) T cell response. In one embodiment, the T cell response is a cytotoxic T cell response.

Another object of the present invention is a method for preparing a subject for an immunotherapy, in particular an adoptive cell therapy, said method comprising administering to the subject in need thereof guanabenz as described hereinabove.

According to one embodiment, the method of the invention is for preparing a subject for an immunotherapy, in particular an adoptive cell therapy, said immunotherapy being used in the treatment of a cancer or an infectious disease.

According to one embodiment, said method for preparing a subject for an immunotherapy, in particular an adoptive cell therapy, comprises administering to the subject in need thereof guanabenz as described hereinabove, wherein a therapeutically effective dose of guanabenz is administered to the subject prior to, concomitantly with, and/or continuously after the administration to the subject of an immunotherapy as described hereinabove.

Another object of the present invention is a method for potentiating an immunotherapy in a subject in need thereof, said method comprising administering to the subject guanabenz as described hereinabove.

According to one embodiment, the method of the invention is for potentiating an immunotherapy in the treatment of a cancer or an infectious disease.

According to one embodiment, said method for potentiating an immunotherapy in a subject in need thereof comprises administering to the subject guanabenz as described hereinabove, wherein a therapeutically effective dose of guanabenz is administered to the subject prior to, concomitantly with, and/or continuously after the administration to the subject of an immunotherapy as described hereinabove.

Another object of the present invention is a method for treating a cancer or an infectious disease in a subject in need thereof, said method comprising administering to the subject an immunotherapy and guanabenz as described hereinabove, wherein said guanabenz is used as a conditioning regimen, thereby preparing the subject for the immunotherapy and/or as an adjuvant for the immunotherapy, thereby potentiating the immunotherapy.

According to one embodiment, said method for treating a cancer or an infectious disease in a subject in need thereof comprises administering to the subject an immunotherapy and guanabenz as described hereinabove, wherein a therapeutically effective dose of guanabenz is administered to the subject prior to, concomitantly with, and/or continuously after the administration to the subject of an immunotherapy as described hereinabove.

Another object of the present invention is a method for treating a cancer in a subject in need thereof, said method comprising administering to the subject an immunotherapy and guanabenz as described hereinabove, wherein said guanabenz is used as a conditioning regimen, thereby preparing the subject for the immunotherapy and/or as an adjuvant for the immunotherapy, thereby potentiating the immunotherapy.

According to one embodiment, said method for treating a cancer in a subject in need thereof comprises administering to the subject an immunotherapy and guanabenz as described hereinabove, wherein a therapeutically effective dose of guanabenz is administered to the subject prior to, concomitantly with, and/or continuously after the administration to the subject of an immunotherapy as described hereinabove.

Another object of the present invention is a method for treating an infectious disease in a subject in need thereof, said method comprising administering to the subject an immunotherapy, in particular a vaccination, and guanabenz as described hereinabove, wherein said guanabenz is used as a conditioning regimen, thereby preparing the subject for the immunotherapy and/or as an adjuvant for the immunotherapy, thereby potentiating the immunotherapy.

According to one embodiment, said method for treating an infectious disease in a subject in need thereof comprises administering to the subject an immunotherapy, in particular a vaccination, and guanabenz as described hereinabove, wherein a therapeutically effective dose of guanabenz is administered to the subject prior to, concomitantly with, and/or continuously after the administration to the subject of an immunotherapy, in particular a vaccination, as described hereinabove.

Another object of the present invention is the use of guanabenz as described hereinabove for the manufacture of a medicament for modulating an immune response in a subject in need thereof.

Another object of the present invention is the use of guanabenz as described hereinabove for the manufacture of a medicament for stimulating or enhancing an immune response, in particular a cellular immune response, in a subject in need thereof.

In one embodiment, the immune response, in particular the cellular immune response, is a T cell response or a NK cell response. In one embodiment, the T cell response is an alpha beta (αβ) T cell response or a gamma delta (γδ) T cell response. In one embodiment, the T cell response is a cytotoxic T cell response.

Another object of the present invention is the use of guanabenz as described hereinabove for the manufacture of a medicament for preparing a subject for a subsequent immunotherapy. In one embodiment, said immunotherapy is a cancer immunotherapy as described hereinabove or a vaccination.

Another object of the present invention is the use of guanabenz as described hereinabove for the manufacture of a medicament for potentiating an immunotherapy in a subject in need thereof. In one embodiment, said immunotherapy is a cancer immunotherapy as described hereinabove or a vaccination.

Another object of the present invention is the use of guanabenz as described hereinabove for the manufacture of a medicament for the treatment of a cancer or of an infectious disease in a subject in need thereof, wherein said medicament is used as a conditioning regimen for an immunotherapy to be subsequently administered to the subject.

Another object of the present invention is the use of guanabenz as described hereinabove for the manufacture of a medicament for the treatment of a cancer or of an infectious disease in a subject in need thereof, wherein said medicament is used as an adjuvant for an immunotherapy administered or to be administered to the subject.

Another object of the present invention is the use of guanabenz as described hereinabove for the manufacture of a medicament for the treatment of a cancer or of an infectious disease in a subject in need thereof in combination with an immunotherapy as described hereinabove.

Another object of the present invention is the use of guanabenz as described hereinabove for the manufacture of a medicament for the treatment of a cancer in a subject in need thereof in combination with an immunotherapy as described hereinabove.

Another object of the present invention is the use of guanabenz as described hereinabove for the manufacture of a medicament for the treatment of an infectious disease in a subject in need thereof in combination with an immunotherapy, in particular a vaccination, as described hereinabove.

Another object of the present invention is a pharmaceutical composition comprising guanabenz as described hereinabove and at least one pharmaceutically acceptable excipient, for use in the treatment of a cancer or of infectious disease in a subject in need thereof, wherein said pharmaceutical composition is used as an adjuvant or as a conditioning regimen for an immunotherapy.

In one embodiment, the pharmaceutical composition for use in the treatment of a cancer or of infectious disease according to the invention comprises guanabenz as described hereinabove, at least one pharmaceutically acceptable excipient, and an immunotherapy as described hereinabove, such as, for example, a checkpoint inhibitor.

Another object of the present invention is a kit-of-parts for use in the treatment of a cancer or of infectious disease in a subject in need thereof comprising a first part comprising a pharmaceutical composition comprising guanabenz as described hereinabove and at least one pharmaceutically acceptable excipient, and a second part comprising an immunotherapy as described hereinabove, such as, for example, a checkpoint inhibitor.

Pharmaceutically acceptable excipients that may be used in the pharmaceutical composition of the invention include, without being not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

Another object of the invention is a medicament comprising guanabenz as described hereinabove, or a pharmaceutical composition as described hereinabove, or a kit-of-parts as described hereinabove, for use in the treatment of a cancer or of an infectious disease in a subject in need thereof, wherein said medicament is used as an adjuvant or as a conditioning regimen for an immunotherapy.

As mentioned hereinabove, guanabenz as described hereinabove is to be administered either simultaneously, separately or sequentially with respect to an immunotherapy as described hereinabove, for which it is used as an adjuvant or as a conditioning regimen.

In one embodiment, guanabenz, the pharmaceutical composition of the invention, the medicament of the invention or the kit-of-parts of the invention will be formulated for administration to the subject. Guanabenz, the pharmaceutical composition, medicament or kit-of-parts of the invention may be administered orally, parenterally, topically, by inhalation spray, rectally, nasally, buccally, vaginally or via an implanted reservoir.

According to one embodiment, guanabenz as described hereinabove is in an adapted form for an oral administration. Thus, in one embodiment, guanabenz is to be administered orally to the subject, for example as a powder, a tablet, a capsule, and the like or as a tablet formulated for extended or sustained release.

In one embodiment, the pharmaceutical composition, medicament or kit-of-parts of the invention is in a form adapted for oral administration. In other words, the pharmaceutical composition, medicament or kit-of-parts of the invention comprises guanabenz and optionally an immunotherapy as described hereinabove in a form adapted for oral administration.

Examples of forms adapted for oral administration include, without being limited to, liquid, paste or solid compositions, and more particularly tablets, tablets formulated for extended or sustained release, capsules, pills, dragees, liquids, gels, syrups, slurries, and suspensions.

According to another embodiment, guanabenz as described hereinabove is in an adapted form for an injection. Thus, in one, guanabenz is to be injected to the subject, by intravenous, intramuscular, intraperitoneal, intrapleural, subcutaneous, transdermal injection or infusion.

In one embodiment, the pharmaceutical composition, medicament or kit-of-parts of the invention is in a form adapted for injection, such as, for example, for intravenous, subcutaneous, intramuscular, intradermal, transdermal injection or infusion. In other words, the pharmaceutical composition, medicament or kit-of-parts of the invention comprises guanabenz and optionally an immunotherapy as described hereinabove in a form adapted for injection, such as, for example, for intravenous, intramuscular, intraperitoneal, intrapleural, subcutaneous, transdermal injection or infusion.

Sterile injectable forms of guanabenz, the pharmaceutical composition or medicament of the invention may be a solution or an aqueous or oleaginous suspension. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to another embodiment, guanabenz as described hereinabove is in an adapted form for a parenteral administration. Thus, in one, guanabenz is to be administered parenterally.

In one embodiment, the pharmaceutical composition, medicament or kit-of-parts of the invention is in a form adapted for parenteral administration. In other words, the pharmaceutical composition, medicament or kit-of-parts of the invention comprises guanabenz and optionally an immunotherapy as described hereinabove in a form adapted for parenteral administration.

According to another embodiment, guanabenz as described hereinabove is in an adapted form for a topical administration. Thus, in one embodiment, guanabenz is to be administered topically.

In one embodiment, the pharmaceutical composition, medicament or kit-of-parts of the invention is in a form adapted for topical administration. In other words, the pharmaceutical composition, medicament or kit-of-parts of the invention comprises guanabenz as described hereinabove and optionally an immunotherapy as described hereinabove in a form adapted for topical administration.

Examples of forms adapted for topical administration include, without being limited to, liquid, paste or solid compositions, and more particularly aqueous solutions, drops, dispersions, sprays, microcapsules, micro- or nanoparticles, polymeric patch, or controlled-release patch, and the like.

According to another embodiment, guanabenz as described hereinabove is in an adapted form for a rectal administration. Thus, in one, guanabenz is to be administered rectally.

In one embodiment, the pharmaceutical composition, medicament or kit-of-parts of the invention is in a form adapted for rectal administration. In other words, the pharmaceutical composition, medicament or kit-of-parts of the invention comprises guanabenz as described hereinabove and optionally an immunotherapy as described hereinabove in a form adapted for rectal administration.

Examples of forms adapted for rectal administration include, without being limited to, suppository, micro enemas, enemas, gel, rectal foam, cream, ointment, and the like.

According to one embodiment, the kit-of-parts of the invention comprises guanabenz as described hereinabove that is in a form adapted for oral administration and an immunotherapy as described hereinabove that is in a form adapted for injection, such as, for example, for intravenous, intramuscular, intraperitoneal, intrapleural, subcutaneous, transdermal injection or infusion. Thus, in one embodiment, the kit-of-parts of the invention comprises guanabenz as described hereinabove that is to be administered orally to the subject and an immunotherapy as described hereinabove that is to be administered by injection to the subject, such as, for example, by intravenous, intramuscular, intraperitoneal, intrapleural, subcutaneous, transdermal injection or infusion.

According to another embodiment, the kit-of-parts of the invention comprises guanabenz as described hereinabove that is in a form adapted for injection, such as, for example, for intravenous, intramuscular, intraperitoneal, intrapleural, subcutaneous, transdermal injection or infusion and an immunotherapy as described hereinabove that is in a form adapted for oral administration. Thus, in one embodiment, the kit-of-parts of the invention comprises guanabenz as described hereinabove to be administered by injection to the subject, such as, for example, by intravenous, intramuscular, intraperitoneal, intrapleural, subcutaneous, transdermal injection or infusion and an immunotherapy as described hereinabove that is that is to be administered orally to the subject.

In one embodiment, guanabenz is to be administered prior to and/or concomitantly with an immunotherapy as described hereinabove. In one embodiment, guanabenz is to be administered between one week and one hour prior to an immunotherapy as described hereinabove, preferably one day prior to an immunotherapy as described hereinabove.

In one embodiment, the immunotherapy is an adoptive cell therapy and guanabenz is to be administered prior to the day(s) or on the same day(s) that the immune cells as described hereinabove are transferred. In another embodiment, the immunotherapy is a checkpoint inhibitor therapy and guanabenz is to be administered prior to the day(s) or on the same day(s) that the checkpoint inhibitor as described hereinabove is administered. In another embodiment, the immunotherapy is a vaccination and guanabenz is to be administered prior to the day(s) or on the same day(s) that the vaccination as described hereinabove is administered.

In one embodiment, guanabenz is to be administered prior to an immunotherapy as described hereinabove, once, twice, three times or more.

In one embodiment, guanabenz is to be administered prior to and/or concomitantly with an immunotherapy as described hereinabove and continuously thereafter.

In one embodiment, guanabenz is to be administered prior to or concomitantly with an immunotherapy as described hereinabove and subsequently for at least 1, 2, 3, 4, 5, 6, 7, 8, 9, or 10 days thereafter. In another embodiment, guanabenz is to be administered prior to or concomitantly with an immunotherapy as described hereinabove and subsequently for at least 1, 2, 3, 4, 5, 6, 7, 8, 9, or 10 weeks thereafter. In another embodiment, guanabenz is to be administered prior to or concomitantly with an immunotherapy as described hereinabove and subsequently for at least 1, 2, 3, 4, 5, 6, 7, 8, 9, or 10 months thereafter.

In one embodiment, the immunotherapy is an adoptive cell therapy and guanabenz is to be administered prior to and/or concomitantly with said adoptive cell therapy and continuously thereafter. In one embodiment, the immunotherapy is an adoptive cell therapy and guanabenz is to be administered prior to and/or concomitantly with said adoptive cell therapy and subsequently for at least 1, 2, 3, 4, 5, 6, 7, 8, 9, or 10 weeks thereafter.

In one embodiment, the immunotherapy is a checkpoint inhibitor therapy and guanabenz is to be administered prior to and/or concomitantly with said checkpoint inhibitor therapy. In one embodiment, the immunotherapy is a checkpoint inhibitor therapy and guanabenz is to be administered prior to and/or concomitantly with said checkpoint inhibitor therapy and continuously thereafter. In one embodiment, the immunotherapy is a checkpoint inhibitor therapy and guanabenz is to be administered prior to or concomitantly with said checkpoint inhibitor therapy and subsequently for at least 1, 2, 3, 4, 5, 6, 7, 8, 9, or 10 weeks thereafter.

In one embodiment, the immunotherapy is a vaccination and guanabenz is to be administered prior to and/or concomitantly with said vaccination. In one embodiment, the immunotherapy is a vaccination and guanabenz is to be administered prior to and/or concomitantly with said vaccination and continuously thereafter. In one embodiment, the immunotherapy is a vaccination and guanabenz is to be administered prior to and/or concomitantly with said vaccination and subsequently for at least 1, 2, 3, 4, 5, 6, 7, 8, 9, or 10 weeks thereafter.

According to one embodiment, a therapeutically effective dose of guanabenz as described hereinabove is to be administered for use in the treatment of a cancer or of an infectious disease in a subject in need thereof, wherein said guanabenz is used as an adjuvant or as a conditioning regimen for an immunotherapy. Thus, in one embodiment, the pharmaceutical composition, medicament or kit-of-parts of the invention comprises a therapeutically effective dose of guanabenz as described hereinabove and optionally a therapeutically effective dose of an immunotherapy as described hereinabove.

It will be understood that the total daily usage of guanabenz will be decided by the attending physician within the scope of sound medical judgment. The specific dose for any particular subject will depend upon a variety of factors such as the cancer or infectious disease to be treated; the age, body weight, general health, sex and diet of the patient; and like factors well-known in the medical arts.

In one embodiment, the subject is a mammal, preferably a human, and the dose of guanabenz, preferably a therapeutically effective dose, is a dose ranging from about 0.01 mg per kilo body weight (mg/kg) to about 30 mg/kg, preferably from about 0.01 mg/kg to about 15 mg/kg, more preferably from about 0.01 mg/kg to about 7 mg/kg. In another embodiment, the subject is a mammal, preferably a human, and the dose of guanabenz, preferably a therapeutically effective dose, is a dose ranging from about 0.01 mg/kg to about 4.5 mg/kg, preferably from about 0.01 mg/kg to about 2 mg/kg, more preferably from about 0.01 mg/kg to about 1 mg/kg.

In one embodiment, the subject is a mammal, preferably a human, and the dose of guanabenz, preferably a therapeutically effective dose, is a dose ranging from about 0.01 mg per kilo body weight per day (mg/kg/day) to about 30 mg/kg/day, preferably from about 0.01 mg/kg/day to about 15 mg/kg/day, more preferably from about 0.01 mg/kg/day to about 7 mg/kg/day. In another embodiment, the subject is a mammal, preferably a human, and the dose of guanabenz, preferably a therapeutically effective dose, is a dose ranging from about 0.01 mg/kg/day to about 4.5 mg/kg/day, preferably from about 0.01 mg/kg/day to about 2 mg/kg/day, more preferably from about 0.01 mg/kg/day to about 1 mg/kg/day.

In one embodiment, the subject is a mammal, preferably a human, and the dose of guanabenz, preferably a therapeutically effective dose, is a dose ranging from about 1 mg to about 2000 mg, preferably from about 1 mg to about 1000 mg, more preferably from about 1 mg to about 500 mg. In one embodiment, the subject is a mammal, preferably a human, and the dose of guanabenz, preferably a therapeutically effective dose, is a daily dose ranging from about 1 mg to about 320 mg, preferably from about 1 mg to about 150 mg. In another embodiment, the subject is a mammal, preferably a human, and the dose of guanabenz, preferably a therapeutically effective dose, is a dose ranging from about 1 to about 100 mg, preferably from about 1 mg to about 70 mg.

In one embodiment, the subject is a mammal, preferably a human, and the dose of guanabenz, preferably a therapeutically effective dose, is a daily dose ranging from about 1 mg to about 2000 mg, preferably from about 1 mg to about 1000 mg, more preferably from about 1 mg to about 500 mg. In one embodiment, the subject is a mammal, preferably a human, and the dose of guanabenz, preferably a therapeutically effective dose, is a daily dose ranging from about 1 mg to about 320 mg, preferably from about 1 mg to about 150 mg. In another embodiment, the subject is a mammal, preferably a human, and the dose of guanabenz, preferably a therapeutically effective dose, is a daily dose ranging from about 1 to about 100 mg, preferably from about 1 mg to about 70 mg.

In one embodiment, the subject is a mammal, preferably a human, and the dose of guanabenz, preferably a therapeutically effective dose, is a dose of at least about 0.01, 0.02, 0.07, 0.15, 0.30, 0.42, 0.55, 0.70 or 0.85 mg/kg. In one embodiment, the subject is a mammal, preferably a human, and the dose of guanabenz, preferably a therapeutically effective dose, is a dose of at least about 0.01, 0.02, 0.07, 0.15, 0.30, 0.42, 0.55, 0.70 or 0.85 mg/kg/day.

In one embodiment, the subject is a mammal, preferably a human, and the dose of guanabenz, preferably a therapeutically effective dose, is a dose of at least about 1, 2, 5, 10, 20, 30, 40, 50 or 60 mg. In one embodiment, the subject is a mammal, preferably a human, and the dose of guanabenz, preferably a therapeutically effective dose, is a daily dose of at least about 1, 2, 5, 10, 20, 30, 40, 50 or 60 mg.

In one embodiment, the subject is a mammal, preferably a human, and the dose of guanabenz, preferably a therapeutically effective dose, is a dose of about 0.057, 0.115, 0.23, 0.46 or 0.92 mg/kg. In one embodiment, the subject is a mammal, preferably a human, and the dose of guanabenz, preferably a therapeutically effective dose, is a dose of about 0.057, 0.115, 0.23, 0.46 or 0.92 mg/kg/day.

In one embodiment, the subject is a mammal, preferably a human, and the dose of guanabenz, preferably a therapeutically effective dose, is a dose of about 4, 8, 16, 32 or 64 mg. In one embodiment, the subject is a mammal, preferably a human, and the dose of guanabenz, preferably a therapeutically effective dose, is a daily dose of about 4, 8, 16, 32 or 64 mg.

In one embodiment, the subject is a mammal, preferably a human, and the dose of guanabenz, preferably a therapeutically effective dose, is a daily dose to be administered in one, two, three or more takes. In one embodiment, the subject is a mammal, preferably a human, and the dose of guanabenz, preferably a therapeutically effective dose, is a daily dose to be administered in one or two takes.

In one embodiment, the cancer to be treated according to the present invention is selected from the group comprising or consisting of acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma.

According to one embodiment, the cancer to be treated according to the present invention is not a gynecological cancer or tumor. In one embodiment, the cancer to be treated according to the present invention is not an ovarian cancer or tumor.

In one embodiment, the cancer to be treated according to the present invention is selected from the group comprising or consisting of acute lymphoblastic leukemia, acute myeloblastic leukemia adrenal gland carcinoma, bile duct cancer, bladder cancer, breast cancer, colorectal cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, pancreatic cancer, prostate cancer, salivary gland cancer, sarcoma, squamous cell carcinoma, testicular cancer, thyroid cancer, urothelial carcinoma, and uveal melanoma.

According to one embodiment, the cancer to be treated according to the present invention is a cancer resistant to cancer immunotherapy as described hereinabove.

Examples of cancer resistant to immunotherapy include, without being limited to, colorectal cancer, pancreatic cancer and prostate cancer.

According to one embodiment, the subject suffering from a cancer to be treated according to the present invention is resistant to cancer immunotherapy as described hereinabove.

In one embodiment, the cancer to be treated according to the present invention is a solid cancer or solid tumor.

As used herein, the term “solid cancer” encompasses any cancer (also referred to as malignancy) that forms a discrete tumor mass, as opposed to cancers (or malignancies) that diffusely infiltrate a tissue without forming a mass.

Examples of solid cancers include, without being limited to, adrenocortical carcinoma, anal cancer, basal cell carcinoma, bile duct cancer, bladder cancer, bone cancer, brain cancer such as glioblastoma or central nervous system (CNS) tumors, breast cancer (such as triple negative breast cancer and inflammatory breast cancer), cervical cancer, uterine cancer, endometrial cancer, colorectal cancer (CRC) such as colon carcinoma, esophageal cancer, eye cancer such as retinoblastoma, gallbladder cancer, gastric cancer (also referred to as stomach cancer), gastrointestinal carcinoma, gastrointestinal stromal tumor (GIST), head and neck cancer (such as for example laryngeal cancer, oropharyngeal cancer, nasopharyngeal carcinoma, or throat cancer), liver cancer such as hepatocellular carcinoma (HCC), Hodgkin's lymphoma, Kaposi sarcoma, mastocytosis, myelofibrosis, lung cancer (such as lung carcinoma, non-small-cell lung carcinoma (NSCLC), and small cell lung cancer), pleural mesothelioma, melanoma such as uveal melanoma, neuroendocrine tumors, neuroblastoma, ovarian cancer, primary peritoneal cancer, pancreatic cancer, parathyroid cancer, penile cancer, pituitary adenoma, prostate cancer such as castrate metastatic prostate cancer, rectal cancer, kidney cancer such as renal cell carcinoma (RCC), skin cancer other than melanoma such as Merkel cell skin cancer, small intestine cancer, sarcoma such as soft tissue sarcoma, squamous-cell carcinoma, testicular cancer, thyroid cancer, and urethral cancer.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is an adoptive cell transfer therapy as described hereinabove, a checkpoint inhibitor therapy as described hereinabove or a vaccination, in particular a therapeutic vaccination, as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is an adoptive cell transfer therapy as described hereinabove or a vaccination, in particular a therapeutic vaccination, as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is an a checkpoint inhibitor therapy as described hereinabove or a vaccination, in particular a therapeutic vaccination, as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is an adoptive cell transfer therapy as described hereinabove or a checkpoint inhibitor therapy as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is an adoptive cell transfer therapy as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is a checkpoint inhibitor therapy as described hereinabove.

In one embodiment, the cancer to be treated according to the present invention is a solid cancer or solid tumor selected from the group comprising or consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma; and the immunotherapy is a vaccination, in particular a therapeutic vaccination, as described hereinabove.

In one embodiment, the solid cancer to be treated according to the present invention is a metastatic solid cancer, i.e., a solid cancer wherein at least one metastatic tumor is observed in addition to the primary tumor.

According to one embodiment, the solid cancer to be treated according to the present invention is a solid cancer or solid tumor with good immunogenicity, i.e., a solid cancer or tumor susceptible to respond to an immunotherapy.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is an adoptive cell transfer therapy as described hereinabove, a checkpoint inhibitor therapy as described hereinabove or a vaccination, in particular a therapeutic vaccination, as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is an adoptive cell transfer therapy as described hereinabove or a vaccination, in particular a therapeutic vaccination, as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is a checkpoint inhibitor therapy as described hereinabove or a vaccination, in particular a therapeutic vaccination, as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is an adoptive cell transfer therapy as described hereinabove or a checkpoint inhibitor therapy as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is an adoptive cell transfer therapy as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is a checkpoint inhibitor therapy as described hereinabove.

In one embodiment, the solid cancer to be treated according to the present invention is a solid cancer or solid tumor with good immunogenicity selected from the group comprising or consisting of melanoma, breast carcinoma, colon carcinoma, renal carcinoma, adrenocortical carcinoma, skin sarcoma, lung carcinoma and liver carcinoma; and the immunotherapy is a vaccination, in particular a therapeutic vaccination, as described hereinabove.

According to one embodiment, the solid cancer to be treated according to the present invention is a solid cancer or solid tumor with low immunogenicity, i.e., a solid cancer or tumor susceptible to be resistant to an immunotherapy.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is an adoptive cell transfer therapy as described hereinabove, a checkpoint inhibitor therapy as described hereinabove or a vaccination, in particular a therapeutic vaccination, as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is an adoptive cell transfer therapy as described hereinabove or a vaccination, in particular a therapeutic vaccination, as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is a checkpoint inhibitor therapy as described hereinabove or a vaccination, in particular a therapeutic vaccination, as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is an adoptive cell transfer therapy as described hereinabove or a checkpoint inhibitor therapy as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is an adoptive cell transfer therapy as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is a checkpoint inhibitor therapy as described hereinabove.

In one embodiment, the solid cancer to be treated according to the present invention is a solid cancer or solid tumor with low immunogenicity selected from the group comprising or consisting of prostate carcinoma, skin sarcoma, fibrosarcoma, glioblastoma, pancreatic carcinoma and testicular teratoma; and the immunotherapy is a vaccination, in particular a therapeutic vaccination, as described hereinabove.

In one embodiment, the solid cancer to be treated according to the present invention is selected from the group comprising or consisting of melanoma, such as uveal melanoma, pancreatic cancer, lung cancer such as lung carcinoma or non-small cell lung cancer, pleural mesothelioma, ovarian cancer, primary peritoneal cancer, prostate cancer, such as castrate metastatic prostate cancer, gastrointestinal carcinoma, breast cancer, liver cancer such as hepatocellular carcinoma, sarcoma, and central nervous system (CNS) tumors. In one embodiment, the solid cancer to be treated according to the present invention is selected from the group comprising or consisting of melanoma, such as uveal melanoma, pancreatic cancer, lung cancer such as lung carcinoma or non-small cell lung cancer, pleural mesothelioma, primary peritoneal cancer, prostate cancer, such as castrate metastatic prostate cancer, gastrointestinal carcinoma, breast cancer, liver cancer such as hepatocellular carcinoma, sarcoma, and central nervous system (CNS) tumors.

In one embodiment, the solid cancer to be treated according to the present invention is selected from the group comprising or consisting of melanoma, Merkel cell skin cancer, Hodgkin's Lymphoma, lung cancer, head and neck cancer, bladder cancer, and kidney cancer.

In one embodiment, the solid cancer to be treated according to the present invention is selected from the group comprising or consisting of cervical cancer, pancreatic cancer, prostate cancer, breast cancer, gastric cancer, and glioblastoma. In one embodiment, the solid cancer to be treated according to the present invention is selected from the group comprising or consisting of pancreatic cancer, prostate cancer, breast cancer, gastric cancer and glioblastoma.

In one embodiment, the solid cancer to be treated according to the present invention is selected from the group comprising or consisting of melanoma, colorectal cancer such as colon carcinoma, lung cancer, head and neck cancer, and bladder cancer

In one embodiment, the solid cancer to be treated according to the present invention is melanoma.

In one embodiment, the solid cancer to be treated according to the present invention is melanoma and the immunotherapy is an adoptive cell transfer therapy as described hereinabove, a checkpoint inhibitor therapy as described hereinabove or a vaccination, in particular a therapeutic vaccination, as described hereinabove.

In one embodiment, the solid cancer to be treated according to the present invention is melanoma and the immunotherapy is an adoptive cell transfer therapy as described hereinabove or a vaccination, in particular a therapeutic vaccination, as described hereinabove.

In one embodiment, the solid cancer to be treated according to the present invention is melanoma and the immunotherapy is a checkpoint inhibitor therapy as described hereinabove or a vaccination, in particular a therapeutic vaccination, as described hereinabove.

In one embodiment, the solid cancer to be treated according to the present invention is melanoma and the immunotherapy is an adoptive cell transfer therapy as described hereinabove or a checkpoint inhibitor therapy as described hereinabove.

In one embodiment, the solid cancer to be treated according to the present invention is melanoma and the immunotherapy is an adoptive cell transfer therapy as described hereinabove.

In one embodiment, the solid cancer to be treated according to the present invention is melanoma and the immunotherapy is a checkpoint inhibitor therapy as described hereinabove.

In one embodiment, the solid cancer to be treated according to the present invention is melanoma and the immunotherapy is a vaccination, in particular a therapeutic vaccination, as described hereinabove.

As defined hereinabove, “infectious disease” as used herein encompasses any disease caused by an infectious agent such as a virus, a bacterium, a fungus or a protozoan parasite.

In one embodiment, said infectious disease is caused by a virus. In other words, in one embodiment, said infectious disease is a viral infection.

In one embodiment, the infectious disease to be treated according to the present invention is caused by a virus and the immunotherapy is a vaccination, such as a preventive or a therapeutic vaccination.

Examples of viruses that may be responsible for a viral infection include, without being limited to, viruses of the families Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepadnaviridae, Hepevirus, Herpesviridae, Leviviridae, Luteoviridae, Mononegavirales, Mosaic Viruses, Nidovirales, Nodaviridae, Orthomyxoviridae, Paramyxoviridae, Papillomaviridae, Picobirnavirus, Picornaviridae, Potyviridae, Reoviridae, Retroviridae, Sequiviridae, Tenuivirus, Togaviridae, Tombusviridae, Totiviridae, and Tymoviridae.

In one embodiment, the infectious disease to be treated according to the present invention is caused by the human immunodeficiency virus (HIV).

In one embodiment, the infectious disease to be treated according to the present invention is caused by an ebolavirus, such as the Zaire ebolavirus.

In one embodiment, said infectious disease is caused by a bacterium. In other words, in one embodiment, said infectious disease is a bacterial infection.

In one embodiment, the infectious disease to be treated according to the present invention is caused by a bacterium and the immunotherapy is a vaccination, such as a preventive or a therapeutic vaccination.

Examples of bacteria that may be responsible for a bacterial infection include, without being limited to, bacteria of the genera Bacillus, including Bacillus anthracis and Lactobacillus; Brucella; Bordetella including B. pertussis and B. bronchiseptica; Camplyobacter; Chlamydia including C. psittaci and C. trachomatis; Corynebacterium including C. diphtheriae; Enterobacter including E. aerogenes; Enterococcus; Escherichia including E. coli; Flavobacterium including F. meningosepticum and F. odoratum; Gardnerella including G. vaginalis; Klebsiella; Legionella including L. pneumophila; Listeria; Mycobacterium including M. tuberculosis, M. intracellulare, M. folluitum, M. laprae, M. avium, M. bovis, M. africanum, M. kansasii, and M. lepraemurium; Neisseria including N. gonorrhoeae and N. meningitides; Nocardia; Proteus including P. mirabilis and P. vulgaris; Pseudomonas including P. aeruginosa; Rickettsia including R. rickettsii; Serratia including S. marcescens and S. liquefaciens; Staphylococcus; Streptomyces including S. somaliensis; Streptococcus, including S. pyogenes; and Treponema.

In one embodiment, the infectious disease to be treated according to the present invention is tuberculosis.

In one embodiment, said infectious disease is caused by a fungus. In other words, in one embodiment, said infectious disease is a fungal infection.

In one embodiment, the infectious disease to be treated according to the present invention is caused by a fungus and the immunotherapy is a vaccination, such as a preventive or a therapeutic vaccination.

Examples of fungi that may be responsible for a fungal infection include, without being limited to, fungi of the genera Aspergillus, Candida, Cryptococcus, Epidermophyton, Microsporum, and Trichophyton.

In one embodiment, said infectious disease is caused by a protozoan parasite. In other words, in one embodiment, said infectious disease is a protozoan infection.

In one embodiment, the infectious disease to be treated according to the present invention is caused by a protozoan parasite and the immunotherapy is a vaccination, such as a preventive or a therapeutic vaccination.

Examples of protozoan parasites that may be responsible for a protozoan infection include, without being limited to, Coccidia, Leishmania, Plasmodium, Toxoplasma and Trypanosoma.

In one embodiment, the infectious disease to be treated according to the present invention is malaria.

### BRIEF DESCRIPTION OF THE DRAWINGS

FIG. 1 shows the effects of guanabenz on T cell function. Mouse TCRP1A CD8⁺ T cells were incubated with guanabenz for 16 hours and co-cultured with L1210-P1A-B7.1 cells as target cells. FIG. 1A is a histogram showing the degranulation of CD8⁺ T cells assessed by FACS detection of CD107a during co-culture. Mouse TCRP1A CD8⁺ T cells were incubated with guanabenz for 24 hours and co-cultured with L1210-P1A-B7.1 cells as target cells. 16 hours after co-culture, the supernatant was collected. FIG. 1B is a histogram showing the secreted IFNγ measured by ELISA in the collected supernatant. Cells from a human anti-WT1 CD8⁺ T cell clone were incubated with guanabenz for 16 hours and co-cultured with target cells pulsed with the WT1 peptide. FIG. 1C is a histogram showing the degranulation of human CD8⁺ T cells measured by FACS detection of CD107a. FIG. 1D is a histogram showing the secretion of IFNγ in the supernatant of the overnight co-culture quantified by ELISA (D).

FIG. 2 shows the effects of guanabenz in T429.11 transplanted tumor bearing mice. The T429.11 transplanted tumor bearing mice received daily injections of guanabenz (5 mg/kg, i.p.) or vehicle (PBS, i.p.) from the day when the tumor size was around 1000 mm³ (day 1) until the day of sacrifice (day 6). FIG. 2A is a graph showing tumor growth in the T429.11 transplanted tumor bearing mice between day 1 and sacrifice (day 6). FIG. 2B is a histogram showing tumor infiltration of CD8⁺ T cells in the T429.11 transplanted tumor bearing mice evaluated by FACS on the day of sacrifice (day 6).

FIG. 3 is a graph showing the tumor growth in TiRP^{+/+} mice that received daily injections of guanabenz (5 mg/kg, i.p.) or vehicle (PBS, i.p.) from the day when the tumor size was around 400 mm³ (day 0) until the day of sacrifice (day 12).

FIG. 4 is a graph showing the tumor size in the immunodeficient Rag1^{-/-} TiRP^{+/+} mice that were injected with 4-OH-Tamoxifen to induce a TiRP tumor. When the tumor reached 500 mm³ (day 0), the tumor bearing mice were randomized and received daily injections of guanabenz (5 mg/kg, i.p.) or vehicle (PBS, i.p.) until the day of sacrifice (day 15).

FIG. 5 shows the effects of guanabenz in TiRP mice that received an adoptive transfer of P1A-specific CD8⁺ T cells. FIG. 5A is a graph showing tumor growth in TiRP mice that received adoptive cell transfer (ACT) of 10 million of P1A-specific activated CD8⁺ T cells and daily injections of guanabenz (5 mg/kg, i.p.) or vehicle (PBS, i.p.) from the day of the ACT, when the tumor size was around 500 mm³ (day 0), until the day of sacrifice (day 18). FIG. 5B is a histogram showing tumor weight in the TiRP mice measured on the day of sacrifice (day 18). Tumor infiltration of P1A-specific CD8⁺ cells evaluated 10 days after ACT by FACS. FIG. 5C is a histogram showing the tumor infiltration of P1A-specific CD8⁺ cells expressed as the percentage of P1A tetramer⁺ CD8⁺ T cells among total living cells in the tumor microenvironment. FIG. 5D is a histogram showing the tumor infiltration of P1A-specific CD8⁺ cells expressed as the percentage of P1A tetramer⁺ cells among total CD8⁺ T cells. FIG. 5E is a histogram showing FACS analysis of apoptosis in tumor infiltrating P1A-specific CD8⁺ TILs evaluated 10 days after ACT. FIG. 5F is a histogram showing FACS analysis of T cell activation marker CD69 in tumor infiltrating P1A-specific CD8⁺ T cells evaluated 10 days after ACT.

FIG. 6 is a histogram showing the tumor infiltration of P1A-specific CD8⁺ T cells evaluated 4 days after ACT of naïve TCRP1A CD8⁺ T cells by FACS in mice that received guanabenz (5 mg/kg, i.p.) or vehicle (PBS, i.p.).

FIG. 7 shows the effect of guanabenz in a mouse immunization model using a vaccine consisting of irradiated L1210-P1A-B7.1 cells. DBA/2 mice received a vaccine consisting of 10⁶ irradiated L1210-P1A-B7.1 cells either alone (immunization) or with 100 μg (5 mg/kg) guanabenz (immunization+guanabenz). Mice that did not receive immunization were included as negative control (control). One week after the immunization, the spleens were collected and the splenocytes isolated. The splenocytes were then stimulated in vitro for four days with L1210-P1A-B7.1 cells at a ratio of 1:1 to expand the P1A-specific CD8⁺ T cells. FIG. 7A is a histogram showing the percentage of P1A-antigen specific CD8⁺ T cells among the total number of CD8⁺ T cells assessed through staining with PE-conjugated HA tetramer and APC-conjugated anti-CD8 antibody four days after the stimulation. After four days in vitro stimulation, the splenocytes were further restimulated with L1210-P1A-B7.1 cells at a ratio of 1:1 overnight. FIG. 7B is a histogram showing the amount of IFNγ secreted by splenocytes was measured by ELISA.

FIG. 8 shows the effect of guanabenz in a model of OVA immunization in mice. C57BL/6J mice were immunized once by intraperitoneal injection of 200 μg of OVA protein adsorbed onto Alhydrogel adjuvant 2% (Sigma). The mice were administered 100 μg of guanabenz two hours before and daily after the immunization (guanabenz). Mice that did not receive immunization were included as a negative control (control). One week after the immunization, the blood and spleen of the immunized mice were collected and cells derived from the blood and spleen were cultured in the presence of 10 μM OVA peptide. FIG. 8A is a histogram showing the immune response evaluated by assessing the percentage of IFNγ producing CD8⁺ T cells among the total number of CD8⁺ T in the cell cultures deriving from the blood of the immunized mice. FIG. 8B is a histogram showing the immune response evaluated by assessing the percentage of IFNγ producing CD8⁺ T cells among the total number of CD8⁺ T in the cell cultures deriving from the spleen of the immunized mice.

FIG. 9 is a graph showing tumor growth in B16F10 transplanted melanoma bearing mice which received either guanabenz alone, anti-PD-1 alone or both guanabenz and anti-PD-1. Mice thus received daily injection of guanabenz (2.5 mg/kg, i.p.) or vehicle (PBS, i.p.) 7 days after tumor inoculation and until sacrifice. Mice then received 4 injections (i.p.) of anti-PD-1 antibody (BioXcell, clone RMP1-14, 200 μg/mouse) or isotype control at 3-day intervals starting 1 day after guanabenz or vehicle administration. Tumor size was monitored every day.

FIG. 10 is a histogram showing the effects of guanabenz on NK cell function. Mouse NK cells were isolated from the splenocytes of TiRP 10B mice using anti-CD49b magnetic beads. After isolation, the NK cells were activated using RMA-S cells. 4 days after activation, NK cells were collected and treated with 20 μM guanabenz for 16 hours. The treated NK cells were then co-cultured with RMA-S cells as target cells. Degranulation of NK cells was assessed by FACS detection of CD107a during co-culture.

FIG. 11 assesses the effects of alprenolol on T cell function and in combination with an adoptive cell transfer. FIG. 11A is a histogram showing the effects of alprenolol on T cell function. Mouse TCRP1A CD8+ T cells were incubated with alprenolol 5 μM or 20 μM as indicated for 16 hours. The treated T cells were then co-cultured with L1210-P1A-B7.1 cells as target cells. Degranulation of CD8+ T cells was assessed by FACS detection of CD107a during co-culture. FIGS. 11B and 11C show the effects of alprenolol in TiRP mice that received an adoptive transfer of P1A-specific CD8+ T cells. FIG. 11B is a graph showing tumor growth in TiRP mice that received adoptive cell transfer (ACT) of 10 million of P1A-specific activated CD8+ T cells and daily injections of alprenolol (5 mg/kg, i.p.) or vehicle (PBS, i.p.) from the day of the ACT, when the tumor size was around 500 mm3 (day 0), until the day of sacrifice (day 10). FIG. 11C is a histogram showing tumor infiltration of P1A-specific CD8+ cells evaluated 7 days after ACT by FACS. The tumor infiltration of P1A-specific CD8+ cells is expressed as the percentage of HA tetramer+CD8+ T cells among total CD45+ cells in the tumor microenvironment. “ns” stands for non-significant.

FIG. 12 assesses the effects of sunitinib on T cell function and in combination with an adoptive cell transfer. FIG. 12A is a histogram showing the effects of sunitinib on T cell function. Mouse TCRP1A CD8+ T cells were incubated with 20 μM guanabenz or different concentration of sunitinib as indicated for 16 hours. The treated T cells were then co-cultured with L1210-P1A-B7.1 cells as target cells. Degranulation of CD8+ T cells was assessed by FACS detection of CD107a during co-culture. FIGS. 12B and 12C show the effects of sunitinib in TiRP mice that received an adoptive transfer of P1A-specific CD8+ T cells. FIG. 12B is a graph showing tumor growth in TiRP mice that received adoptive cell transfer (ACT) of 10 million of P1A-specific activated CD8+ T cells and daily administration of sunitinib (20 mg/kg) or vehicle (PBS) by oral gavage from the day of the ACT, when the tumor size was around 500 mm3 (day 0), until the day of sacrifice (day 10). FIG. 12C is a histogram showing the effects of sunitinib in TiRP mice that received an adoptive transfer of P1A-specific CD8+ T cells. Mice in the sunitinib group received sunitinib daily by oral gavage at a dose of 20 mg/kg. The tumor infiltration of P1A-specific CD8⁺ cells is expressed as the percentage of P1A tetramer+CD8+ T cells among total living cells in the tumor microenvironment. “ns” stands for non-significant.

### EXAMPLES

The present invention is further illustrated by the following examples.

### Example 1

Materials and Methods

Material

Mice

TiRP mice: TiRP mice have been created by crossing Ink4a/Arf^{flox/flox} mice with mice carrying a transgenic construct controlled by the tyrosinase promoter and driving the expression of H-Ras^{12V} and Trap1a which encodes a MAGE-type tumor antigen P1A; the promoter is separated from the coding region by a stop cassette made of a floxed self-deleting CreER (Huijbers et al., 2006, Cancer Res 66, 3278-3286). Those mice were backcrossed to a B10.D2 background and bred to homozygosity. TCRP1A mice heterozygous for the H-2Ld/P1A35-43-specific TCR transgene were kept on the B10.D2; Rag1^{-/-} background (Shanker et al., 2004, J Immunol 172, 5069-5077). All mice used in this study were produced under specific pathogen free (SPF) conditions at the animal facility of the Ludwig Institute for Cancer Research. All the rules concerning animal welfare have been respected according to the 2010/63/EU Directive. All procedures were performed with the approval of the local Animal Ethical Committee, with reference 2015/UCL/MD/15.

TiRP-derived T429.11 transplanted melanoma model: T429.11 clone was derived from an induced Amela TiRP tumor referred to as T429. It was cloned from the T429 induced melanoma primary tumor line. Two million of T429.11 tumor cells were injected subcutaneously into recipient mice for tumor establishment (Zhu et al, Nat Commun 2017, 10; 8(1):1404).

Cells

Mouse TCRP1A CD8⁺ T cells: P1A-specific (TCRP1A) CD8⁺ T cells were isolated from spleens and lymph nodes of TCRP1A mice using anti-mouse CD8a (Ly-2) MicroBeads (Miltenyi Biotec).

Human anti-WT1 CD8⁺ T cells: a cDNA construct encoding the recombinant TCR against WT1 126-134 peptide presented by HLA A2 was introduced into PBMCs (peripheral blood mononuclear cells) from a hemochromatosis patient and TCR⁺ CD8⁺ T cells were then sorted and cloned using a WT1₁₂₆₋₁₃₄ HLA2 tetramer. The T cells were then cultured using irradiated T2 cells pulsed with WT1₁₂₆₋₁₃₄ peptide and irradiated allogeneic EBVB cells in the presence of IL2 (100 U/mL).

Methods

In Vitro T Cell Function

CD107 cytotoxicity assay (Degranulation assay): TCRP1A CD8⁺ T cells were plated at 50,000 cells per well in a 96 U plate with different concentrations of guanabenz and incubated at 37° C. overnight prior to their use in the assay on the next day. On the day of the assay, the culture supernatant was removed from the cells and the cells were washed once with complete medium to remove the drug. The target cells L1210-P1A-B7.1 were added to the wells at a ratio of 1:1. Control wells containing either only T cells or target cells were also included for each plate. CD107a-APC was added to each well at the same time as addition of the target cells. The plate was then incubated at 37° C. for 90 minutes. At the end of the incubation period the cells were harvested and washed once with PBS. They were stained with anti-mouse CD8-Bv421 antibody for 15 minutes. The cells were then washed and resuspended in PBS and analyzed using FACS Fortessa flow cytometer. Human CD8⁺ T cell degranulation was evaluated in a similar way. To induce CD8⁺ T cell degranulation, T2 cells loaded with synthetic WT1₁₂₆₋₁₃₄ peptide (10⁶ T2 cells were incubated for 1 hour in 200 μL Optimem medium with 100 μmol/L synthetic peptide at 37° C.) were used as target cells.

IFNγ secretion assay: CD8⁺ T cells were plated at 50,000 cells per well in a 96 U plate with different concentration of guanabenz in full medium supplemented with IL2 and incubated at 37° C. overnight prior to their use in the assay on the next day. On the day of the assay, the culture supernatant was removed from the cells and the cells were washed once with complete medium to remove the drug completely. The target cells L1210-P1A-B7.1 or T2 cells loaded with WT1₁₂₆₋₁₃₄ peptide were added to the wells at a ratio of 1:1. Control wells containing either only T cells or effector cells were also included for each plate. The plate was then incubated at 37° C. overnight. At the end of the incubation period the supernatant was collected and the amount of IFNγ was measured by ELISA according to manufacturer's instruction (R&D).

Tumor Induction with 40H-Tamoxifen

A fresh solution of 40H-Tamoxifen was prepared by dissolving 40H-Tamoxifen (Imaginechem) in 100% ethanol and mineral oil (ratio 1:9) followed by 30-min sonication, and injected subcutaneously (2 mg/200 μL per mouse) in the neck area of gender-matched 7 weeks old TiRP mice. Tumor appearance was monitored daily and tumors were measured three times/week. Tumor volume (in mm³) was calculated by the following formula: Volume=width²×length/2. Tumor-bearing TiRP mice were randomized based on the tumor size when average volume was 400 mm³, 500 mm³ or 1000 mm³ as indicated.

Guanabenz Administration

T cells were incubated with guanabenz (10, 20, or 40 μM) for 16 h to 24 h as indicated. Mice received a daily intra-peritoneal injection of guanabenz (5 mg/kg) or vehicle (PBS) from the day of randomization (and the day of ACT when applicable) until the day of sacrifice.

Adoptive Cell Transfer with TCRP1A CD8⁺ T Cells

For the adoptive cell transfer (ACT), P1A-specific (TCRP1A) CD8⁺ T cells were isolated from spleens and lymph nodes of TCRP1A mice as described hereinabove, and stimulated in vitro by co-culture with irradiated (10.000 rads) L1210-P1A-B7.1 cells (Gajewski et al., 1995, J Immunol 154, 5637-5648) at 1:2 ratio (0.5×10⁵ CD8⁺ T cells and 10⁵ L1210-P1A-B7.1 cells per well in 48-well plates) in IMDM (GIBCO) containing 10% fetal bovine serum supplemented with L-arginine (0.55 mM, Merck), L-asparagine (0.24 mM, Merck), glutamine (1.5 mM, Merck), betamercaptoethanol (50 μM, Sigma), 50 UmL⁻¹ penicillin and 50 mg mL⁻¹ streptomycin (Life Technologies). Four days later, TCRP1A CD8⁺ T cells were purified on a Lymphoprep gradient (StemCell) and 10⁷ living cells were injected intravenously in 200 μL PBS in TiRP-tumor bearing mice on the day of randomization.

Results

In Vitro Effects of Guanabenz on T Cell Function

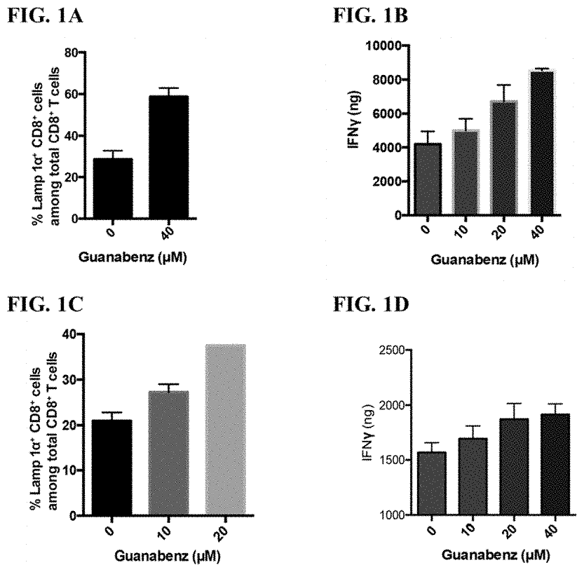

Murine P1A-specific (TCRP1A) CD8⁺ T cells, co-cultured with L1210-P1A cells expressing the HA antigen, were incubated with guanabenz for 16 hours. T cell function following antigen recognition was assessed by detecting degranulation and secretion of interferon gamma (IFNγ). As shown on FIG. 1A-B, incubation of murine T cells with guanabenz increased both the T cell degranulation (FIG. 1A) and the secretion of IFNγ (FIG. 1B). Similar results were obtained with human anti-WT1 CD8⁺ T cells co-cultured with target cells pulsed with the WT1 peptide and incubated with guanabenz (FIG. 1C-D). The results shown on FIG. 1 thus demonstrate that guanabenz is able to increase T cell function in vitro.

In Vivo Effects in TiRP-Derived T429.11 Transplanted Melanoma Model

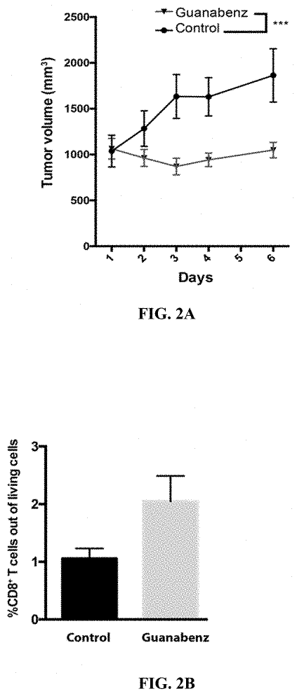

The T429.11 transplanted melanoma model was previously shown not to respond to anti-PD-1 and anti-CTLA4 therapy (Zhu et al., Nature communications. Nov. 10 2017; 8(1):1404). T429.11 transplanted tumor bearing mice received daily injections of guanabenz (5 mg/kg, i.p.) or vehicle (PBS, i.p.) from the day when the tumor size was around 1000 mm³ (day 1) until the day of sacrifice (day 6). As shown on FIG. 2A, guanabenz inhibited tumor growth even though the administration of guanabenz was started at a late stage (tumor at a size of 1000 mm³) and in absence of anti-PD-1 and anti-CTLA4 therapy. As shown on FIG. 2B, the decreased tumor growth was accompanied with an increased tumor infiltration of CD8⁺ T cells.

In Vivo Effects in TiRP Melanoma Model

The TiRP is a genetically engineered mouse melanoma model based on the tamoxifen-driven Cre-mediated expression of H-Ras^{G12V} and deletion of Ink4A/Arf in melanocytes, concomitantly with the expression of a specific tumor antigen of the MAGE-type, called HA. The TiRP model is characterized by tumors that are locally aggressive and insensitive to immunotherapies such as adoptive cell transfer (ACT). In particular, the TiRP model does not respond to the ACT of activated CD8⁺ T cells specific for the HA antigen (TCRP1A CD8⁺ T cells). The lack of response is explained by the fact that the transferred TCRP1A CD8⁺ T cells undergo apoptosis and disappear from the tumors in a few days. One of the main factors responsible for the immunoresistance of the TiRP tumors is the tumor enrichment in polymorphonuclear myeloid-derived suppressor cells (PMN-MDSC) that are able to induce apoptosis of tumor-infiltrating lymphocytes (TILs), e.g., tumor-infiltrating TCRP1A CD8⁺ T cells, through the Fas/Fas-ligand axis (Zhu et al., Nature communications. Nov. 10 2017; 8(1):1404).

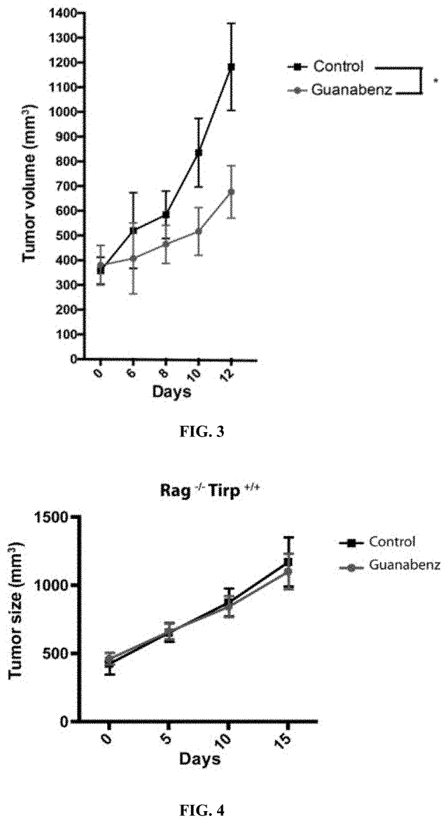

TiRP-tumor bearing mice were administered daily injections of guanabenz from the day when the tumor size was around 400 mm3 until the day of sacrifice. As shown on FIG. 3, guanabenz displayed inhibitory effect on TiRP tumor growth, presumably by boosting the endogenous anti-tumor immune response.

As a control, tumors were induced in immunodeficient TiRP mice lacking T cells due to the deletion of the Rag1 gene (Rag 1^{-/-} TiRP^{+/+} mice). When the tumor reached about 500 mm³, the TiRP-tumor bearing mice received daily injections of guanabenz until the day of sacrifice. Strikingly, guanabenz was no longer effective and did not induce a decrease in the TiRP tumor growth when compared to the control (FIG. 4). This result thus confirms that the inhibitory effect of guanabenz on tumor growth is immune-mediated.

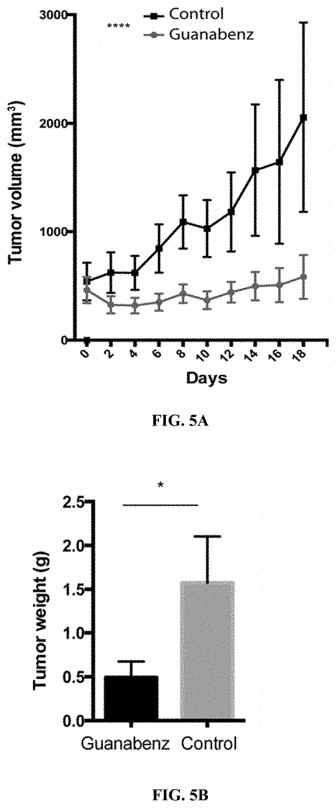

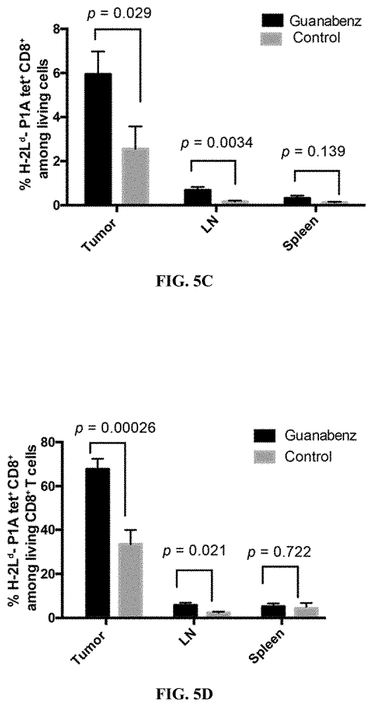

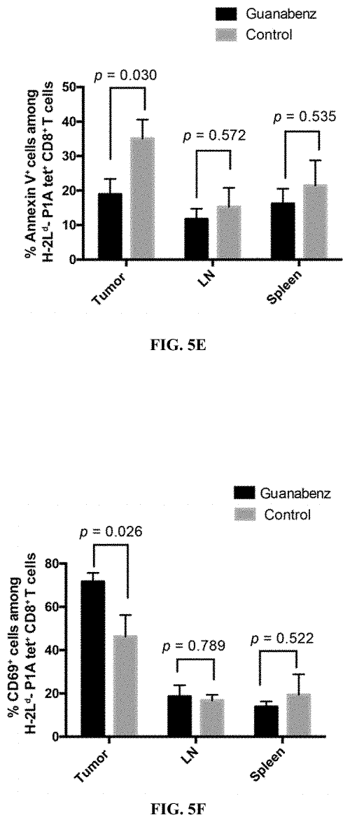

TiRP-tumor bearing mice were also administered guanabenz and an adoptive cell transfer (ACT) of 10 million of P1A-specific activated CD8⁺ T cells. Daily injections of guanabenz were thus administered from the day of the ACT, when the tumor size was around 500 mm³, until the day of sacrifice. As shown on FIG. 5, guanabenz strongly sensitized immune-resistant autochthonous melanoma tumors (TiRP) to adoptive cell transfer (ACT). Both tumor growth (FIG. 5A) and tumor weight on the day of sacrifice (FIG. 5B) were significantly decreased with guanabenz used with an ACT, as compared to an ACT alone. Following guanabenz administration, tumor infiltration of CD8⁺ T cells was increased (FIG. 5C-D) and apoptosis of the adoptively transferred CD8⁺ T cells was reduced (FIG. 5E). Moreover, the tumor infiltrated CD8⁺ T cells were also more active in the mice that received guanabenz, as shown with the increased percentage of CD69⁺ P1A-specific CD8⁺ T cells (FIG. 5F). These results demonstrate that guanabenz improves the therapeutic efficacy of adoptive cell transfer.

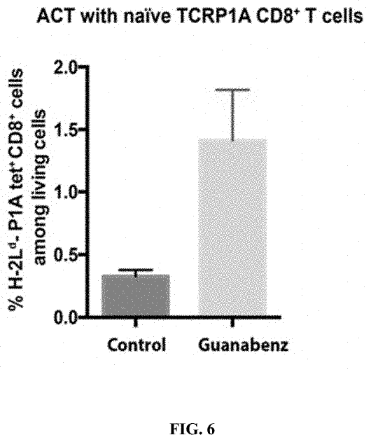

In the preceding experiments, before being transferred to TiRP-tumor bearing mice, the P1A-specific CD8⁺ T were pre-activated in vitro by co-culture with irradiated L1210-P1A-B7.1 cells as described hereinabove. Indeed, previous studies showed that when naïve TCRP1A CD8⁺ T cells are transferred to TiRP-tumor bearing mice, said naïve CD8⁺ T cells fail to be primed properly (Soudja et al., Cancer research. May 1, 2010; 70(9):3515-3525). TiRP-tumor bearing mice were administered an adoptive cell transfer (ACT) of 2 million of naïve P1A-specific CD8⁺ T cells and daily injections of guanabenz from the day of the ACT. As shown on FIG. 6, in TiRP mice that received guanabenz, the tumor infiltration of P1A-specific CD8⁺ T cells 4 days after the ACT was significantly increased when compared to the control. Thus, when the mice were administered guanabenz, the transferred naïve CD8⁺ T cells were properly primed and populated the tumor. These data suggest that Guanabenz might work as single immunotherapeutic like immune checkpoint inhibitors that release the immune inhibitory brakes and restore the T cell anti-tumor function.

### Example 2

Materials and Methods

Material

Mice

DBA/2 mic and C57BL/6J mice were used in immunization experiments.

Methods

Immunization

Immunization with irradiated L1210-P1A-B7.1 tumor cells: DBA/2 mice received a vaccine consisting of 1 million irradiated L1210-P1A-B7.1 cells expressing the P1A antigen either alone or with 100 μg (5 mg/kg) guanabenz. When administered, guanabenz was given 1 hour before the immunization and daily after the immunization. Mice that did not receive immunization were included as negative control.

Immunization with Ovalbumin: C57BL/6J mice were immunized once by intraperitoneal (i.p.) injection of 200 ug of OVA protein adsorbed onto Alhydrogel adjuvant 2% (Sigma) These mice were administered 100 μg (5 mg/kg) of guanabenz 2 hours before the immunization and daily after the immunization. Mice that did not receive immunization were included as negative control.

Evaluation of Immune Response

Intracellular IFNγ staining: one week after the immunization, the blood and spleen of each mouse were collected. Cells derived from the spleen and blood of each immunized mouse were cultured for 1 hour in 96 well U bottomed plates at 37° C. in the presence of 10 μM OVA peptide. Brefeldin A (10 μg/mL) was added to each well and the cells were incubated for an additional 4 hours. The immune response of each mouse was evaluated by measuring the amount of IFNγ producing CD8⁺ T cells using antibodies against CD8 and IFNγ. Samples were then examined by FACS Fortessa flow cytometry.

Tetramer staining: one week after the immunization, the spleen of each mouse was collected. Isolated splenocytes from the immunized mice were cultured and restimulated with L1210-P1A-B7.1 cells at a ratio of 1:1. Four days after restimulation, P1A-antigen specific CD8⁺ T cells were stained with PE-conjugated P1A tetramer and APC-conjugated anti-CD8 antibody.

Interferon gamma secretion: one week after the immunization, the spleen of each mouse was collected. Splenocytes from the immunized mice were restimulated with L1210-P1A-B7.1 cells at a ratio of 1:1. Four days after restimulation, the splenocytes were collected and plated at 50,000 cells per well in 96 U plate. L1210-P1A-B7.1 cells were added to each well as target cells at a ratio of 1:1. The plate was then incubated at 37° C. overnight. At the end of the incubation, the supernatant from the cells were collected and the amount of secreted IFNγ was measured by ELISA according to the manufacturer's instruction (R&D).

Results

DBA/2 mice were immunized with a vaccine consisting of 10⁶ irradiated L1210-P1A-B7.1 cells (L1210 leukemia cells expressing P1A and B7-1), either alone or with 100 μg (5 mg/kg) guanabenz. When administered, guanabenz was given 1 hour before the vaccine and daily after the immunization. Mice that did not receive immunization were included as negative controls.

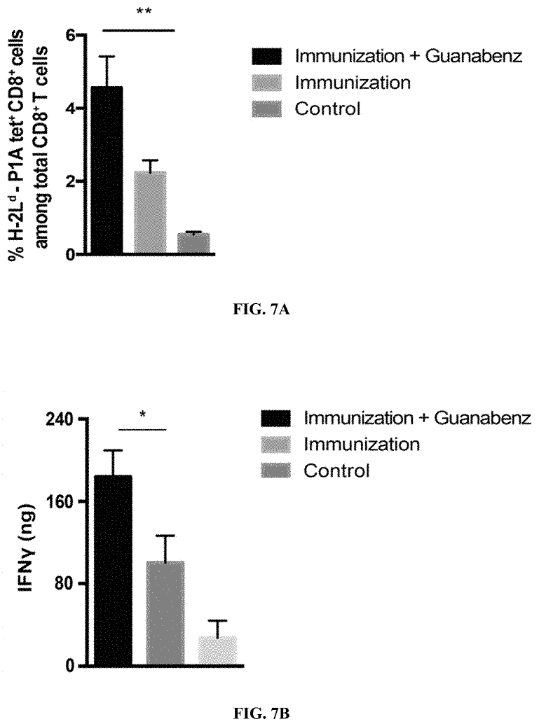

One week after the immunization, the mice were sacrificed and the spleens collected. The isolated splenocytes were restimulated with L1210-P1A-B7.1 cells at a ratio of 1:1. Four days after the restimulation, P1A-antigen specific CD8⁺ T cells were stained with PE-conjugated P1A tetramer and APC-conjugated anti-CD8 antibody. The percentage of P1A-antigen specific CD8⁺ T cells among the total number of CD8⁺ T cells comprised within the splenocyte culture was thus determined after restimulation with the P1A-antigen. As shown on FIG. 7A, there was a significant increase in the percentage of P1A-antigen specific CD8⁺ T cells after restimulation with the P1A-antigen when guanabenz was administered with the vaccine, as compared to the negative control (i.e., mice that were not immunized) and also as compared to the immunization alone (i.e., vaccine without guanabenz). In the context of an immunization against HA, the administration of guanabenz thus resulted in an increase in the number of P1A-antigen specific CD8+ T cells.

The immune response was further confirmed by evaluating the IFNγ secretion from the spleen-derived CD8⁺ T cells. The isolated splenocytes were plated at 50,000 cells per well in 96 U plate. L1210-P1A-B7.1 cells were added to each well as target cells at a ratio of 1:1. The plate was then incubated at 37° C. overnight. At the end of the incubation, the supernatant from the cells was collected and the amount of secreted IFNγ was measured by ELISA. As shown on FIG. 7B, there was a significant increase in the splenocyte IFNγ secretion after their activation with L1210-P1A-B7.1 cells when guanabenz was administered with the vaccine, as compared to the negative control (i.e., mice that were not immunized) and also as compared to the immunization alone (i.e., vaccine without guanabenz). In the context of an immunization against HA, the administration of guanabenz thus resulted in an increase in the function of splenocytes activated by the presence of P1A.

Taken all together, these results demonstrate that in a mouse immunization model using a vaccine consisting of 10⁶ irradiated L1210-P1A-B7.1 cells, guanabenz acts as an adjuvant by enhancing the cellular immune response against HA, notably by inducing an increase in the number of P1A-antigen specific CD8⁺ T cells and by enhancing the function of splenocytes activated in the presence of HA.

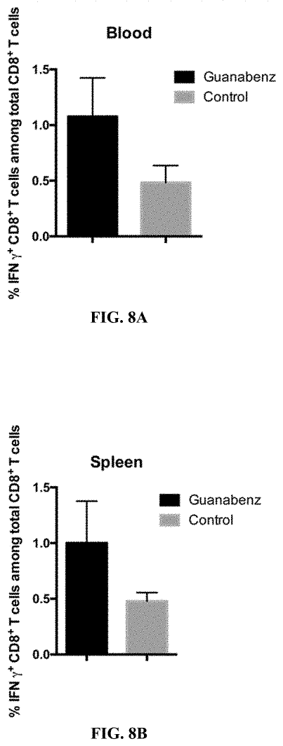

The effect of guanabenz as an adjuvant was also assessed in a model of OVA immunization in mice (FIG. 8). C57BL/6J mice were immunized once by intraperitoneal (i.p.) injection of 200 μg of OVA protein adsorbed onto Alhydrogel adjuvant 2% (Sigma). The mice received 100 μg (5 mg/kg) of guanabenz 2 hours before and daily after the immunization. Mice that did not receive immunization were included as negative control. One week after the immunization, the blood and spleen of each mouse were collected. The immune response was evaluated by assessing the percentage of IFNγ producing CD8⁺ T cells among the total number of CD8⁺ T in cell cultures derived from the blood (FIG. 8A) or the spleen (FIG. 8B) of the mice. As shown on FIG. 8, there was a significant increase in the percentage of IFNγ producing CD8⁺ T cells after their activation with OVA peptide when guanabenz was administered with the vaccine, as compared to the negative control (i.e., mice that were not immunized). These results thus show that in an OVA immunization model, guanabenz acts as an adjuvant by enhancing the cellular immune response against OVA, notably by stimulating the T cell function.

### Example 3

Materials and Methods

Material

Mice

Gender and age matched CD57BL/6 wild-type mice were used in the B16F10 transplanted melanoma model.

Methods

Tumor Induction and Mice Treatments

Gender-matched, 7-9 weeks old CD57BL/6 wild-type mice were subcutaneously injected with 1 million of B16F10 tumor cells. Mice were randomized based on the tumor size one week after the injection of tumor cells. 7 days after tumor inoculation, daily injections of guanabenz (2.5 mg/kg, i.p.) or vehicle (PBS, i.p.) were administered to the mice. Mice then received 4 injections (intraperitoneally also referred to as i.p.) of a dose of 200 μg/mouse anti-PD-1 antibody (BioXcell, clone RMP1-14) or RatIgG2a isotype (clone 2A3, Bio-X-Cell) at 3-day intervals starting 1 day after guanabenz or vehicle administration.

Results

The effect on tumor growth of guanabenz in association with a PD-1 inhibitor was assessed in B16F10 transplanted melanoma bearing mice.

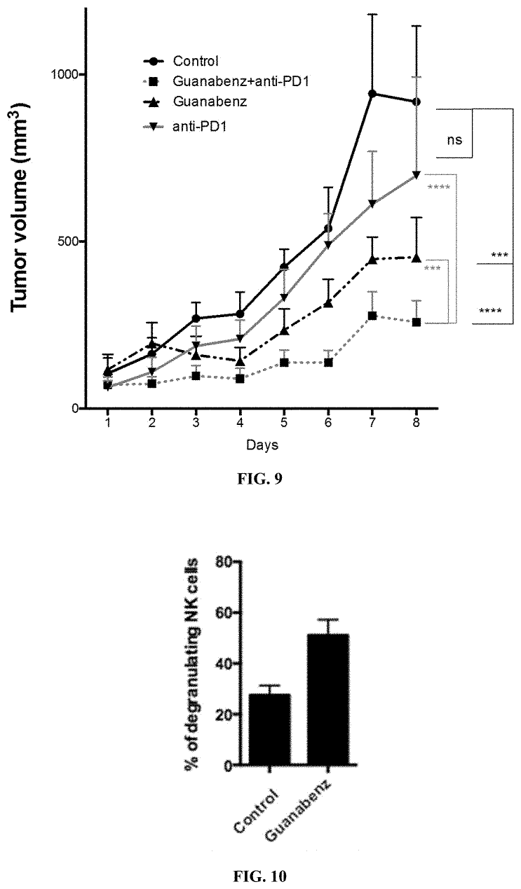

As shown on FIG. 9, as compared to the control condition (administration of PBS and isotype), the growth of B16F10 melanoma tumors was not significantly affected by the administration of an anti-PD-1 antibody alone whereas the administration of guanabenz alone did significantly reduce the growth of B16F10 melanoma tumors.

Strikingly, the combined administration of guanabenz and an anti-PD-1 antibody induced a significant reduction of the growth of B16F10 melanoma tumors as compared to the control condition (administration of PBS and isotype), as compared to the administration of the anti-PD-1 antibody alone but also as compared to the administration of guanabenz alone. Thus, the effect of the combined administration of guanabenz and an anti-PD-1 antibody was significantly greater than the effect of guanabenz alone and the effect the anti-PD-1 antibody alone.

These results show that guanabenz and the anti-PD-1 antibody acted in synergy in reducing the growth of B16F10 melanoma tumors, with guanabenz potentiating the action of the anti-PD-1 antibody. These results show that guanabenz is able to improve the therapeutic efficacy of a checkpoint inhibitor.

### Example 4

Materials and Methods

Material

Cells

Mouse NK cells: murine NK cells were isolated from mouse splenocytes using anti-CD49b magnetic beads.

Methods

In Vitro NK Cell Function

Murine NK cells were isolated from mouse splenocytes using anti-CD49b magnetic beads and were activated in vitro by co-incubation with irradiated RMA-S cells. 4 days after activation, they were collected and plated at 50,000 cells per well in a 96 U plate with 20 μM of guanabenz and incubated at 37° C. overnight prior to their use in the assay on the next day. On the day of the assay, the culture supernatant was removed from the cells and the cells were washed once with complete medium to remove the drug. The target cells (RMA-S cells) were added to the wells at a ratio of 1:1. Control wells containing either only NK cells or target cells were also included for each plate. CD107a-APC was added to each well at the same time as addition of the target cells. The plate was then incubated at 37° C. for 90 minutes. At the end of the incubation period the cells were harvested and washed once with PBS. They were stained with anti-mouse CD49b-PE antibody for 15 minutes. The cells were then washed and resuspended in PBS and analyzed using FACS Fortessa flow cytometer.

Results

In Vitro Effects of Guanabenz on NK Cell Function

Murine NK cells were isolated from mouse splenocytes and activated in vitro by co-incubation with irradiated RMA-S cells. Four days after activation, the NK cells were incubated with guanabenz (20 μM) at 37° C. for 16 hours. NK cell function was assessed by detecting degranulation following co-culture of the NK cell with the target cells (RMA-S cells). As shown on FIG. 10, incubation of murine NK cells with guanabenz significantly increased the NK cell degranulation. The results shown on FIG. 10 thus demonstrate that guanabenz is able to increase NK cell function in vitro.

### Example 5

The effects of alprenolol and sunitinib were assessed using the same models that were used to assess the effects of guanabenz.

Alprenolol is a beta-adrenergic receptor antagonist, used as an antihypertensive, anti-anginal, and anti-arrhythmic agent.

Sunitinib is a small molecule, receptor tyrosine kinase (RTK) inhibitor that has been described as being able to act as an adjuvant for a T-cell mediated cancer immunotherapy (Kujawski et al., Cancer Res. 2010 Dec. 1; 70(23):9599-610).

Materials and Methods

Material

Mice

TiRP mice: TiRP mice are created as described hereinabove (see Example 1).

Cells

Mouse TCRP1A CD8⁺ T cells: P1A-specific (TCRP1A) CD8⁺ T cells were isolated as described hereinabove (see Example 1).

Methods

In Vitro T Cell Function

CD107 cytotoxicity assay (Degranulation assay): the assay was carried out as described hereinabove (see Example 1) with different 20 μM guanabenz or different concentrations of alprenolol or sunitinib, as indicated.

Guanabenz Administration

T cells were incubated with 20 μM guanabenz for 16 h as indicated.

Alprenolol Administration

T cells were incubated with alprenolol (5 or 20 μM) for 16 h as indicated.

Mice received a daily intra-peritoneal injection of alprenolol (5 mg/kg) or vehicle (PBS) from the day of ACT until the day of sacrifice.

Sunitinib Administration

T cells were incubated with sunitinib (0.1, 0.3, 0.8, 2.5 or 4 μM) for 16 h as indicated. Mice received a daily dose of sunitinib (20 mg/kg) or vehicle (PBS) by oral gavage from the day of ACT until the day of sacrifice.

Results

In Vitro Effects of Alprenolol on T Cell Function

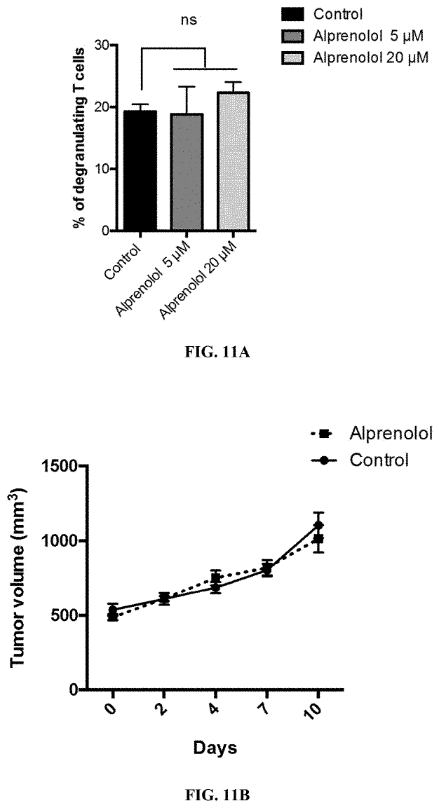

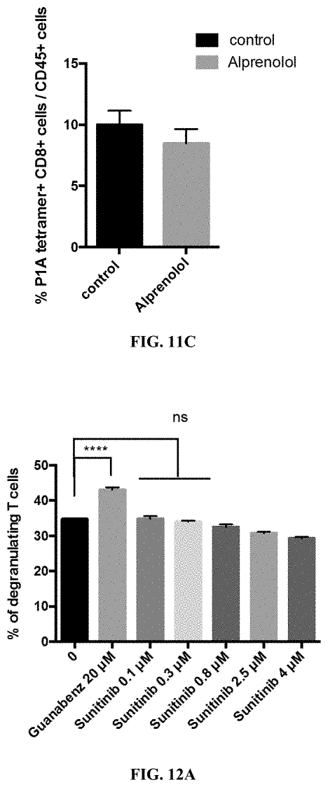

Murine P1A-specific (TCRP1A) CD8⁺ T cells, co-cultured with L1210-P1A cells expressing the HA antigen, were incubated with alprenolol 5 μM or 20 μM for 16 hours. T cell function following antigen recognition was assessed by detecting degranulation. As shown on FIG. 11A, incubation of murine T cells with alprenolol did no significantly increase the T cell degranulation when compared to the control (administration of PBS).

It thus appears that, contrary to what was observed with guanabenz (see FIG. 1), alprenolol is not able to increase T cell function in vitro, either at a concentration of 5 μM or at a concentration of 20 μM.

In Vivo Effects of Alprenolol in TiRP Melanoma Model

TiRP-tumor bearing mice, obtained as described hereinabove (see Example 1), were administered alprenolol and an adoptive cell transfer (ACT) of 10 million of P1A-specific activated CD8⁺ T cells. Daily injections of alprenolol 5 mg/kg (intraperitoneal or in short i.p.) were thus administered from the day of the ACT (day 0), when the tumor size was around 500 mm³, until the day of sacrifice (day 10). As shown on FIG. 11B, tumor growth was not significantly decreased with an ACT administered with alprenolol, as compared to an ACT administered alone (control condition corresponding to the administration of PBS). Accordingly, following alprenolol administration, tumor infiltration of CD8⁺ T cells was not increased (FIG. 11C). These results show that alprenolol does not improve the therapeutic efficacy of adoptive cell transfer, contrary to what was observed with guanabenz (see FIG. 5).

In Vitro Effects of Sunitinib on T Cell Function

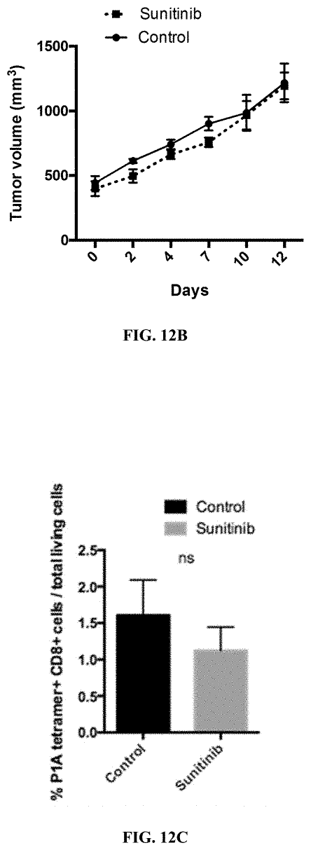

Murine P1A-specific (TCRP1A) CD8⁺ T cells, co-cultured with L1210-P1A cells expressing the HA antigen, were incubated with 20 μM guanabenz or sunitinib (0.1 μM, 0.3 μM, 0.8 μM, 2.5 μM or 4 μM) for 16 hours. T cell function following antigen recognition was assessed by detecting degranulation. As shown on FIG. 12A, incubation of murine T cells with sunitinib did no significantly increase the T cell degranulation when compared to the control (administration of PBS). By contrast, incubation of murine T cells with 20 μM guanabenz did significantly increase the T cell degranulation when compared to the control.

It thus appears that, contrary to what is observed with guanabenz, sunitinib is not able to increase T cell function in vitro, either at a low concentration (i.e., 0.1 μM) or at a high concentration (i.e., 4 μM).

In Vivo Effects of Alprenolol in TiRP Melanoma Model

TiRP-tumor bearing mice, obtained as described hereinabove (see Example 1), were administered sunitinib and an adoptive cell transfer (ACT) of 10 million of P1A-specific activated CD8⁺ T cells. Daily oral gavage of sunitinib 20 mg/kg was thus administered from the day of the ACT (day 0), when the tumor size was around 500 mm³, until the day of sacrifice (day 10). As shown on FIG. 12B, tumor growth was not significantly decreased with an ACT administered with sunitinib, as compared to an ACT administered alone (control condition corresponding to the administration of PBS). Accordingly, following sunitinib administration, tumor infiltration of CD8⁺ T cells was not increased (FIG. 12C). These results show that sunitinib does not improve the therapeutic efficacy of adoptive cell transfer, contrary to what was observed with guanabenz (see FIG. 5).

1-15. (canceled)

16. A method for treating a cancer or an infectious disease in a subject in need thereof, said method comprising administering to the subject an immunotherapy and guanabenz.

17. The method according to claim 16, wherein guanabenz is to be administered as an adjuvant for the immunotherapy.

18. The method according to claim 16, wherein guanabenz is to be administered as a conditioning regimen for the immunotherapy, a conditioning regimen being a therapy for preparing the subject for the immunotherapy.

19. The method according to claim 16, wherein cancer is a solid cancer selected from the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma and pancreatic carcinoma.

20. The method according to claim 16, wherein the infectious disease is caused by a virus, a bacterium, a fungus or a protozoan parasite.

21. The method according to claim 16, wherein guanabenz is to be administered prior to and/or concomitantly with the immunotherapy.

22. The method according to claim 16, wherein guanabenz is to be administered at a dose ranging from about 0.01 mg per kilo body weight (mg/kg) to about 15 mg/kg.

23. The method according to claim 16, wherein said immunotherapy comprises an adoptive transfer of immune cells.

24. The method according to claim 23, wherein said immune cells are T cells or natural killer (NK) cells.

25. The method according to claim 23, wherein said immune cells are CAR T cells or CAR NK cells.

26. The method according to claim 23, wherein said immune cells are autologous immune cells.

27. The method according to claim 23, wherein said immune cells are CD8⁺ T cells.

28. The method according to claim 16, wherein said immunotherapy comprises at least one T-cell agonist.

29. The method according to claim 16, wherein said immunotherapy comprises a checkpoint inhibitor.

30. The method according to claim 28, wherein said checkpoint inhibitor is selected from the group comprising inhibitors of PD-1 such as pembrolizumab, nivolumab, cemiplimab, tislelizumab, spartalizumab, ABBV-181 and JNJ-63723283, inhibitors of PD-L1 such as avelumab, atezolizumab and durvalumab, inhibitors of CTLA-4 such as ipilimumab and tremelimumab, and any mixtures thereof.

31. The method according to claim 16, wherein said immunotherapy comprises a vaccination.

32. The method according to claim 16, wherein said immunotherapy comprises at least one antibody.

33. The method according to claim 16, wherein said immunotherapy comprises at least one oncolytic virus.

34. The method according to claim 16, wherein said immunotherapy comprises at least one cytokine.

35. The method according to claim 16, wherein the dose of guanabenz is ranging from about 0.01 mg/kg/day to about 30 mg/kg/day.
